(19) United States
(12) Patent Application Publication
(10) Pub. No.: US 2023/0313322 A1
(43) Pub. Date: Oct. 5, 2023

(54) ON-SITE VIRAL INACTIVATION AND RNA PRESERVATION OF GARGLE AND SALIVA SAMPLES COMBINED WITH DIRECT ANALYSIS OF SARS-COV-2 RNA ON MAGNETIC BEADS

(71) Applicant: The Governors of the University of Alberta, Edmonton (CA)

(72) Inventors: Xiaochun Chris LE, Edmonton (CA); Xing-Fang Li, Edmonton (CA); D. Lorne Tyrrell, Edmonton (CA); Yanming Liu, Edmonton (CA); Teresa Kumblathan, Edmonton (CA)

(21) Appl. No.: 18/164,027

(22) Filed: Feb. 3, 2023

Related U.S. Application Data
(60) Provisional application No. 63/306,639, filed on Feb. 4, 2022.

(51) Int. Cl. C12Q 1/70; C12N 15/10; C12Q 1/6806; C12Q 1/686

(52) U.S. Cl. CPC C12Q 1/70; C12N 15/1013; C12Q 1/6806; C12Q 1/686; C12Q 2600/16

(57) ABSTRACT

On-site viral inactivation and RNA preservation of gargle and saliva samples combined with direct analysis of SARS-COV2 RNA on magnetic beads.

### FIELD

The present disclosure relates generally to on-site viral inactivation and RNA preservation of gargle and saliva samples combined with direct analysis of SARS-COV2 RNA on magnetic beads.

### BACKGROUND

The gold standard method for the detection of Severe Acute Respiratory Syndrome Coronavirus 2 (SARS-CoV-2) typically involves three steps: collection of upper respiratory specimens using nasopharyngeal swabs (NPS), extraction of viral RNA using commercial kits, and detection of RNA using reverse transcription quantitative polymerase chain reaction (RT-qPCR). The insertion of the NPS into the nasopharynx is uncomfortable,’ and irritation during NPS sampling may induce sneezing or coughing and expel viral particles, resulting in potential contamination and transmission of SARS-CoV-2. The detection sensitivity is affected by variations in the time of collection, clinical course of infection, and sampling skills.²⁻⁵

Saliva and gargle samples have been used as alternatives to NPS for the detection of SARS-CoV-2 (Tables 1-4). Respiratory viruses can enter the oral cavity from the lower respiratory tract and nasopharynx. Saliva and gargle then collects viral shedding within the oral cavity.⁶ SARS-associated coronavirus was previously detected in throat wash and saliva for diagnosis of SARS infection in the 2003 outbreak.⁷ There are several advantages in using saliva and gargle samples for the detection of SARS-CoV-2. Saliva and gargle collection is fast, easy, and conveniently done at home. Self-collection of samples at home avoids the overcrowding of testing centers, minimize the spread of disease, and reduce the need for healthcare professionals.⁸⁻¹¹ Two studies demonstrated that 87-88% of patients were willing to self-collect saliva samples¹² and 99% willing to self-collect gargle samples.9 Saliva and gargle collection is more comfortable and suitable for various population groups, especially the elderly and children. The use of saliva and gargle samples can mitigate the shortage of swabs and viral transport media due to the global demand for NPSs.^{1,} ¹³ SARS-CoV-2 in saliva and gargle samples have adequate stability for at least seven days at a variety of storage temperatures,^{14,} ¹⁵ which allows ample time for sample collection and delivery to a testing laboratory. In addition to clinical diagnosis, saliva and gargle have been considered for repeated sampling, sample banking, mass testing for asymptomatic infections, testing in rural and resource-limited locations, and point-of-care testing (POCT) applications (examples shown in Tables 1-3).

Nevertheless, the use of saliva and gargle samples for the detection of SARS-CoV-2 faces several analytical challenges compared to the use of NPS. First, the levels of SARS-CoV-2 in saliva and gargle samples could be lower than those in NPS. The false negative rate of SARS-CoV-2 in saliva samples is slightly higher than that of NPS samples.¹⁶ Furthermore, compared to saliva samples, the level of SARS-CoV-2 in gargle samples could be further diluted by the saline solution used for sample collection. Second, compared to NPS, saliva and gargle samples contain more complicated sample matrix, affecting RNA extraction and subsequent analysis. Saliva samples are heterogeneous and viscous, often containing sputum. Commercial RNA extraction kits used in conjunction with NPS samples cannot be directly used for saliva samples because it is difficult to efficiently extract RNA and remove RT-qPCR inhibitors from saliva. Researchers have attempted to dilute saliva samples prior to RNA extraction to lower inhibitor concentration, but dilution decreases detection sensitivity.^{9,} ¹⁷ Other studies have tried to bypass RNA extraction and attempted to detect SARS-Cov-2 directly in saliva using RT-qPCR. However, these attempts were not successful as they showed substantially lower positive results [60% (95% Cl: 49%-70%)] than studies which had an extraction step [89% (95% Cl: 83%-94%)].¹⁶ Furthermore, on-site inactivation of virus-containing samples during self-collection helps to avoid potential transmission risks. However, the released viral RNA are prone to digestion by enzymes present in saliva and gargle samples.

### SUMMARY

In one aspect there is provided on-site viral inactivation and RNA preservation of gargle and saliva samples combined with direct analysis of SARS-COV2 RNA on magnetic beads.

In one aspect there is provided a method of detecting SARS-CoV-2, or variants thereof, in a sample, comprising:
i) combining a sample with a buffer to obtain a mixture, the buffer comprising: a chaotropic agent, a reducing agent, a surfactant, and a serine protease,
ii)- heating said mixture to obtain a heated mixture;
iii)- subjecting the heated mixture to a centrifugation step to obtain a supernatant comprising RNA;
iiii) combining the supernatant with a magnetic bead suspension, such that at least a portion of the RNA within the supernatant binds to the magnetic beads, the magnetic bead suspension obtained by suspension of magnetic beads in a bead-binding buffer to obtain RNA-bound magnetic beads,
   washing said RNA-bound magnetic beads to obtained washed beads; and
   conducting a reverse-transcription polymerase chain reaction (RT-PCR) assay on the washed beads using primers to amplify SARS-CoV-2 or variants thereof.

In one example, wherein said chaotropic agent is guanidinium isothiocyanate, said reducing agent is mercaptoethanol, said surfactant is Triton X-100, and/or said serine protease is proteinase K.

In one example, wherein (i) said buffer comprises about 6 M guanidinium isothiocyanate, about 3% 2-mercaptoethanol, about 2.5% Triton X-100, and about 170 ng/µL proteinase K.

In one example, wherein (i) said buffer further comprises about 17 ng/µL glycogen.

In one example, wherein said bead-binding buffer comprises about 20 mM Tris-HCl pH 8.0, about 2 M NaCl, about 36% PEG 8000, and about 2 mM EDTA.

In one example, wherein (ii) heating said mixture comprises said mixture at 55° C. for about 10 minutes

In one example, wherein (iii) said centrifugation step comprises subjecting the heated mixture to a centrifugation of 13000xg for 5 minutes

In one example, in (iiii) further comprising washing said RNA-bound magnetic beads, comprising:
adding ethanol to said RNA-bound magnetic beads mixing for about 10 seconds at room temperature,
collecting said RNA-bound magnetic beads by centrifugation at 13000xg for about 2 minutes to obtain collected RNA-bound magnetic beads,
washing said collected RNA-bound magnetic beads with 75% ethanol followed by air drying for 5 minutes to obtain dried RNA-bound magnetic beads, and
resuspending said dried RNA-bound magnetic beads RNase-free water containing 200 ng/µL of Proteinase K inhibitor.

In one example, wherein the sample is saliva.

In one example, wherein the sample is a gargle solution using water or saline.

In one example, wherein the sample is from a human.

In one aspect there is provided an RNA Preservation buffer, comprising:

a chaotropic agent, a reducing agent, a surfactant, and a serine protease.

In one example, said chaotropic agent is guanidinium isothiocyanate, said reducing agent is mercaptoethanol, said surfactant is Triton X-100, and/or said serine protease is proteinase K.

In one example, said buffer comprises 6 M guanidinium isothiocyanate, 3% 2-mercaptoethanol, 2.5% Triton X-100, 170 ng/µL proteinase K.

In one example, said buffer further comprises about 17 ng/µL glycogen.

In one aspect there is provided a kit for detecting SARS-CoV-2, or variants thereof, in a sample, comprising:
a) 6 M guanidinium isothiocyanate, 3% 2-mercaptoethanol, 2.5% Triton X-100, 170 ng/µL of proteinase K, and 17 ng/µL of glycogen, and
b) a container.

In one example, further comprising a bead-binding buffer comprising 20 mM Tris-HCl pH 8.0, 2 M NaCl, 36% PEG-8000, and 2 mM EDTA.

### BRIEF DESCRIPTION OF THE FIGURES

Embodiments of the present disclosure will now be described, by way of example only, with reference to the attached Figures.

FIG. 1. Schematic showing the overall process and benefits of an integrated method for sensitive detection of SARS-CoV-2 in gargle and saliva. The method consists of three components: self-collection of gargle or saliva, with simultaneous inactivation of the virus and preservation of the viral RNA; concentration of RNA from the gargle or saliva samples onto magnetic beads; and RT-qPCR analysis of the specific genes of SARS-CoV-2 RNA on the magnetic beads without the need for elution.

FIG. 2. (A) Evaluation of SARS-CoV-2 RNA stability in gargle samples collected from a SARS-CoV-2 positive patient and FIG. 2B(B) the spiked gargle samples. All the samples were treated with the VIP buffer. The gargle samples collected from the SARS-CoV-2 positive patient were either analyzed immediately or stored for one week before analysis. The gargle samples spiked with 65 or 390 copies of the viral RNA were treated with the VIP buffer and stored for up to eight weeks .B). Triplicate aliquots of the stored samples were analyzed each week. The error bars represent one standard deviation of Ct values from triplicate measurements. NTC denotes no template control (negative control). ND indicates no detectable SARS-CoV-2 RNA.

FIG. 3. Determination of the sensitivity of the VIP-Mag-RT-qPCR method by analysis of samples containing different concentrations of SARS-CoV-2 viral RNA. Different amounts of SARS-CoV-2 viral RNA were spiked into 200 µL pooled negative gargle sample, and the spiked samples were processed using the VIP-Mag method followed by RT-qPCR detection. The numbers on top of each bar indicate the number of samples tested positive in 5 replicates. The error bars represent one standard deviation of Ct values from analyses of five replicate samples. NTC denotes no template control, and ND indicates no detectable SARS-CoV-2 RNA.

FIG. 4. Comparison of the VIP-Mag method to the standard QIAamp Viral RNA Mini Kit used with RT-qPCR for the detection of SARS-CoV-2. With the QIAamp Viral RNA Mini Kit method, either 5 µL or 13.5 µL of the extracted RNA was used for RT-qPCR detection of SARS-CoV-2. With the VIP-Mag method, the magnetic beads capturing all RNA were directly analyzed. The error bars represent one standard deviation from triplicate measurements. NTC indicates no template control. ND indicates no detectable SARS-CoV-2 RNA.

FIG. 5. Monitoring of the SARS-CoV-2 RNA levels in saliva and gargle samples collected by a NPS-confirmed SARS-CoV-2 positive patient. Multiple samples were collected every day for 25 days starting from the fifth day since positive NPS test. The error bars represent one standard deviation from the triplicate measurements.

FIG. 6. Monitoring of the SARS-CoV-2 RNA levels in gargle samples collected by another NPS-confirmed SARS-CoV-2 positive patient. Multiple samples were collected every day for a month starting from the third day since positive NPS test. The error bars represent one standard deviation from the triplicate measurements. ND indicates no detectable SARS-CoV-2 RNA.

FIG. 7. Performance evaluation of the VIP-Mag-RT-qPCR method on pooled samples. Three SARS-CoV-2 positive samples (Ct 29.2, 31.4, and 32.2) were each diluted by 8, 16, and 32 times with gargle samples that were pooled from ten SARS-CoV-2 negative volunteers. Each diluted and undiluted samples were analyzed in triplicate using the VIP-Mag-RT-qPCR method. The error bars represent one standard deviation from the triplicate measurements. NTC denotes no template control, and ND indicates no detectable SARS-CoV-2 RNA.

FIG. 8. On-site sample inactivation and RNA preservation enables sensitive detection of SARS-CoV-2 in saliva and gargle. The technique has diverse applications, from community surveillance to public health compliance.

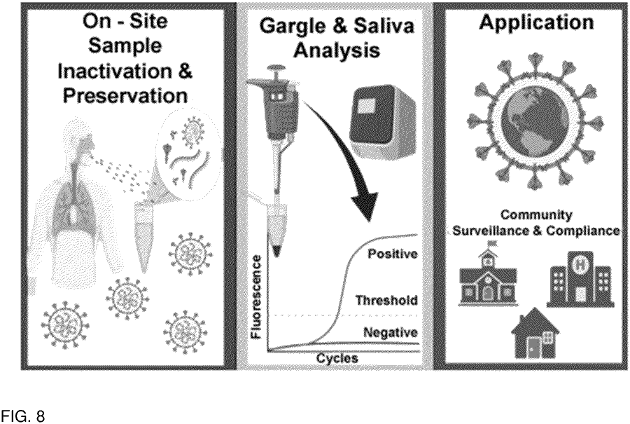

FIGS. 9A and 9B. (FIG. 9A) Detection of SARS-CoV-2 RNA in tap water gargle and saline gargle samples. Viral RNA were added to SARS-CoV-2 negative tap water gargle or saline gargle samples and detected after storage at room temperature for 2 h. (FIG. 9B) Detection of SARS-CoV-2 RNA in three tap water gargle and three saline gargle samples collected from a SARS-CoV-2 positive patient. The error bars represent one standard deviation of triplicate measurements. NTC (no template control) is negative control. ND indicates no detectable SARS-CoV-2 RNA.

FIG. 10. Detection of SARS-CoV-2 RNA in gargle samples treated with glycogen or carrier RNA. The gargle samples contained either 390 or 3900 copies of SARS-CoV-2 RNA. The error bars represent one standard deviation of triplicate measurements. NTC (no template control) is negative control. ND indicates no detectable SARS-CoV-2 RNA.

FIG. 11. Detection of SARS-CoV-2 RNA in saliva and gargle samples treated with either freshly prepared VIP buffer or the VIP buffer stored at room temperature for six months. The saliva and gargle samples each contained 390 copies of viral RNA. The error bars represent one standard deviation of triplicate measurements. NTC stands for no template control. ND indicates no detectable SARS-CoV-2 RNA.

FIG. 12. Detection of SARS-CoV-2 RNA in the absence or the presence of commercial beads. Solid Phase Reversible Immobilization select beads (SPRIselect beads) and silica-based beads (TurboBeads) were tested. Ten or 20 µL of SPRIselect or TurboBeads were washed three times with RNase-free water and then added into a sample of 2000 copies of SARS-CoV-2 RNA. The samples were analyzed using RT-qPCR. The error bars represent one standard deviation of triplicate measurements. NTC denotes no template control. ND indicates no detectable SARS-CoV-2 RNA. PC indicates positive control. Similar Ct values from the reactions containing SARS-CoV-2 RNA with or without the presence of SPRIselect beads indicate that the SPRIselect beads did not affect the RT-qPCR detection.

FIG. 13. Comparison of different concentrations of PEG 8000 in the beads-binding buffer for concentrating SARS-CoV-2 RNA. The error bars represent one standard deviation of triplicate measurements. NTC (no template control) is negative control containing all reagents including 36% PEG. ND indicates no detectable SARS-CoV-2 RNA.

FIG. 14. Detection of 65, 390, or 3900 copies of SARS-CoV-2 RNA in saliva and gargle samples using the VIP-Meg-RT-qPCR method. The error bars represent one standard deviation of triplicate measurements. NTC (no template control) is negative control. ND indicates no detectable SARS-CoV-2 RNA.

FIG. 15. Recovery of viral RNA from RNase-free water and pooled SARS-CoV-2 negative gargle samples. RNase-free water and pooled gargle samples from healthy volunteers were each spiked with 65, 390, or 3900 copies of SARS-CoV-2 RNA. The samples were analyzed using the VIP-Meg-RT-qPCR method. The error bars represent one standard deviation of triplicate measurements. NTC (no template control) is negative control. ND indicates no detectable SARS-CoV-2 RNA.

FIG. 16. Standard curve from the RT-qPCR analysis of the N1 gene segment (CDC). The log values of the numbers of pure SARS-CoV-2 RNA are plotted against the corresponding Ct values. E represents PCR efficiency which was calculated using the equation: E = -1+10(-1/slope), where slope refers to the slope of the standard curve. This standard curve was used to quantify the amounts of SARS-CoV-2 RNA in samples.

FIG. 17. SARS-CoV-2 RNA levels in saliva samples collected four times a day on five days. The samples were collected from the first SARS-CoV-2 positive patient volunteer from the sixth to the tenth day. Lines represent the mean of duplicates, shown individually as symbols. NTC (no template control) is negative control.

FIG. 18. SARS-CoV-2 RNA levels in gargle samples collected four times a day on seven days. The gargle samples were collected from the second SARS-CoV-2 positive patient volunteer from the fourth to the 11^{th} day. Lines represent the mean of duplicates, shown individually as symbols. NTC (no template control) denotes negative control.

### DETAILED DESCRIPTION

Coronaviruses are a large family of viruses which cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS).

Recently identified is the 2019 novel coronavirus (SARS-CoV-2 (SCoV2)/COVID-19).

Severe Acute Respiratory Coronavirus 2 (SARS-CoV-2), the causal agent of COVID-19, was characterized as a pandemic by the World Health Organization (WHO) in March 2020 and has triggered an international public health emergency.

A number of variants of SARS-CoV-2 have been identified.

Variants are viruses that have changed or mutated. Variants are common with coronaviruses. A variant form may confer an evolutionary advantage or disadvantage relative to a progenitor form or may be neutral.

In one aspect, there is provided a method of detecting SARS-COV-2, or variants thereof, in a sample, comprising:
i) combining a sample comprising saliva with a buffer to obtain a mixture, the buffer comprising: 6 M guanidinium isothiocyanate, 3% 2-mercaptoethanol, 2.5% Triton X-100, 170 ng/µL of proteinase K, and 17 ng/µL of glycogen,
ii)- heating said mixture to obtain a heated mixture;
iii)- subjecting the heated mixture to a centrifugation step to obtain a supernatant comprising RNA;
iiii) combining the supernatant with a magnetic bead suspension, such that at least a portion of the RNA within the supernatant binds to the magnetic beads, the magnetic bead suspension obtained by suspension of magnetic beads in a bead-binding buffer comprising 20 mM Tris-HCl pH 8.0, 2 M NaCl, 36% PEG-8000, and 2 mM EDTA, to obtain RNA-bound magnetic beads,
   washing said RNA-bound magnetic beads to obtained washed beads; and
   conducting a reverse-transcription polymerase chain reaction (RT-PCR) assay on the washed beads using primers to amplify SARS-CoV-2 or variants thereof.

In some examples, the bead-binding buffer contains between 0.5 M and 4 M NaCl, preferably 2 M. In some examples, the bead-binding buffer contains between 18% to 45% PEG-8000, preferably 36%.

In one example, wherein (ii) heating said mixture comprises said mixture at 55° C. for about 10 minutes

In one example, wherein (iii) said centrifugation step comprises subjecting the heated mixture to a centrifugation of 13000xg for 5 minutes

In one example, in (iiii) further comprising washing said RNA-bound magnetic beads, comprising:
adding ethanol to said RNA-bound magnetic beads mixing for about 10 seconds at room temperature,
collecting said RNA-bound magnetic beads by centrifugation at 13000xg for about 2 minutes to obtain collected RNA-bound magnetic beads,
washing said collected RNA-bound magnetic beads with 75% ethanol followed by air drying for 5 minutes to obtain dried RNA-bound magnetic beads, and
resuspending said dried RNA-bound magnetic beads RNase-free water containing 200 ng/µL of Proteinase K inhibitor.

In one example, wherein the sample is saliva.

In one example, wherein the sample is a gargle solution using water or saline.

In one example, wherein the sample is from a subject, preferably a human.

The term “subject”, as used herein, refers is to an individual. Non-limiting examples of a subject may include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject may be a mammal such as a primate or a human.

In some examples, the sample may comprise biological cells.

As used herein, “sample” or “biological sample” refers to a composition containing a material to be detected, such as a target polynucleotide.

The term “polynucleotide”, as used herein, refers to a single or double stranded polymer composed of nucleotide monomers.

The term “amplicon” as used herein refers to a polynucleotide DNA or RNA molecule that is the product of an enzymatic or chemical-based amplification event or reaction. An amplicon may be single or double stranded. Enzymatic or chemical-based amplification events or reactions include, without limitation, the polymerase chain reaction (PCR), including RT-PCR.

In one aspect, there is provided an RNA Preservation buffer, comprising:

a chaotropic agent, a reducing agent, a surfactant, and a serine protease.

The chaotropic agent in the RNA Preservation buffer facilitates inactivation of the SARS-CoV-2 virus. In non-limiting examples, the chaotropic agent is a guanidinium or guanidine salt, such as quanidinium isothiocyanate or guanidine hydrochloride. In further examples, the RNA Preservation buffer contains quanidinium isothiocyanate at a concentration of between 4 M to 6 M, preferably 6 M.

The surfactant in the RNA Preservation buffer also facilitates inactivation of the SARS-CoV-2 virus. In non-limiting examples, the surfactant is Triton X-100 or Tergitol-15-S-9. In further examples, the RNA Preservation buffer contains between 0.5-10% Triton X-100, preferably 2.5% Triton X-100.

The reducing agent in the RNA Preservation buffer is selected for its ability to break disulfide bonds within RNase and proteins in samples. In non-limiting examples, the reducing agent is 2-mercaptoethanol, dithiothreitol, or tris(2-carbosyethyl)phosphine). In further examples, the RNA Preservation buffer contains between 1% to 3% 2-mercaptoethanol.

In some examples, the serine protease in the RNA Preservation buffer is a broad spectrum serine protease. In other examples, the serine protease is a subtilisin-type serine protease such as Proteinase K or Proteocut K. In further examples, the RNA Preservation buffer contains between 70 to 340 ng/µL Proteinase K, preferably 170 ng/µL.

Optionally, the RNA Preservation buffer may contain a RNA carrier to enhance the recovery of low amounts of RNA. In some examples, the RNA carrier is glycogen or Carrier RNA. In further examples, the RNA Preservation buffer contains up to 68 ng/µL glycogen, preferably 17 ng/µL.

In one aspect, there is provided a kit for detecting SARS-COV-2, or variants thereof, in a sample, comprising:
a) 6 M guanidinium isothiocyanate, 3% 2-mercaptoethanol, 2.5% Triton X-100, 170 ng/µL of proteinase K, and 17 ng/µL of glycogen, and
b) a container.

In one example, the kit further comprises a bead-binding buffer comprising 20 mM Tris-HCl pH 8.0, 2 M NaCl, 36 % PEG-8000, and 2 mM EDTA.

In the case of identification of a sample with SARS-CoV-2, the subject from which the sample originates may be treated for SARS-CoV-2. Such treatments are known to the skilled worker.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein, such as a fungal or protozoan infection. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of exposure to a pathogen). Treatment may also be continued after symptoms have resolved, for example, to delay or prevent recurrence.

As used herein, in one embodiment the term “about” refers to ±10%. In another embodiment, the term “about” refers to ±9%. In another embodiment, the term “about” refers to ±9%. In another embodiment, the term “about” refers to ±8%. In another embodiment, the term “about” refers to ±7%. In another embodiment, the term “about” refers to ±6%. In another embodiment, the term “about” refers to ±5%. In another embodiment, the term “about” refers to ±4%. In another embodiment, the term “about” refers to ±3%. In another embodiment, the term “about” refers to ±2%. In another embodiment, the term “about” refers to ±1%.

### EXAMPLES

### Abstract

Nasopharyngeal swab (NPS) samples are commonly used for the detection of SARS-CoV-2 and diagnosis of COVID-19. As an alternative, self-collection of saliva and gargle samples minimizes transmission to healthcare workers and relieves the pressure of resources and healthcare personnel during the pandemic. This study aimed to develop an enhanced method enabling simultaneous viral inactivation and RNA preservation during on-site self-collection of saliva and gargle samples. Our method involves the addition of saliva or gargle samples to a newly formulated viral inactivation and RNA preservation (VIP) buffer, concentration of the viral RNA on magnetic beads, and detection of SARS-CoV-2 using reverse transcription quantitative polymerase chain reaction directly from the magnetic beads. This method has a limit of detection of 25 RNA copies per 200 µL gargle or saliva sample and providing 9-111 times higher sensitivity than the viral RNA preparation kit recommended by the United States Centers for Disease Control and Prevention. The integrated method was successfully used to analyze more than 200 gargle and saliva samples, including the detection of SARS-CoV-2 in 123 gargle and saliva samples collected daily from two NPS-confirmed positive SARS-CoV-2 patients throughout the course of their infection and recovery. The VIP buffer is stable at room temperature for at least six months. SARS-CoV-2 RNA (65 copies/ 200 µL sample), is stable in the VIP buffer at room temperature for at least three weeks. The on-site inactivation of SARS-CoV-2 and preservation of the viral RNA enables self-collection of samples, reduces risks associated with SARS-CoV-2 transmission, and maintains the stability of the target analyte.

### Experimental Section

To confront these challenges and facilitate the use of saliva and gargle samples for the detection of SARS-CoV-2, we introduce an integrated sampling and analysis approach that enables simultaneous sample collection, SARS-CoV-2 inactivation, RNA preservation, and sensitive detection. A carefully formulated viral inactivation and RNA preservation (VIP) buffer serves the dual purpose of inactivating SARS-CoV-2 and preserving the released viral RNA. The approach and reagents used for the self-collection of saliva and gargle samples are compatible with the subsequent magnetic bead-based RNA capture to concentrate the viral RNA. Direct analysis of the viral RNA on the beads without requiring an elution step maximizes the sample input amount and enhances the sensitivity of the assay. The analytical strategy and integrated protocol make it feasible to achieve the objective of enhancing safety during sample collection and delivery while maintaining RNA integrity, and thus enable efficient concentration and sensitive detection of SARS-CoV-2 in saliva and gargle samples.

### Materials and Reagents

Proteinase K, QIAamp® Viral RNA Mini Kit, and Buffer RLT Lysis buffer were purchased from QIAgen (Germantown, MD, USA). TaqPath™ 1-Step RT-qPCR Master Mix, CG, UltraPure™ guanidine isothiocyanate, RNA-grade glycogen, and THE RNA Storage Solution were bought from ThermoFisher Scientific (Carlsbad, CA, USA). CDC N1 and N2 primer-probes 2019-nCoV RUO kit was bought from Integrated DNA Technologies (Coralville, IA, USA). Proteinase K Inhibitor tetrapeptidyl chloromethyl ketone was bought from MilliporeSigma (Oakville, Ontario, Canada). RNasin® Plus RNase Inhibitor was bought from Promega (Madison, WI, USA). SPRIselect magnetic beads were bought from Beckman Coulter (Brea, CA, USA). Silica coated TurboBeads® were ordered from TurboBeads®Bio (Zurich, Switzerland). Polyethylene glycol (PEG) Bio Ultra 8000 was bought from MilliporeSigma. 2-Mercaptoethanol (2-ME), biotechnology grade, was bought from BioShop Canada (Burlington, Ontario, Canada). Pseudovirus (SARS-CoV-2 RNA targets in a noninfectious viral coat) solution, AccuPlex™ SARS-CoV-2 Verification Panel-Full Genome, was bought from Sera Care, LGC (Milford, MA, USA). QuickExtract™ RNA Extraction Kit and QuickExtract™ Plant DNA Extraction Solution were bought from Lucigen (Middleton, WI, USA). Purified SARS-CoV-2 RNA was provided by our colleagues in the Department of Medical Microbiology and Immunology at the University of Alberta.

### Viral Inactivation and RNA Preservation (VIP) Buffer

The VIP buffer was formulated to contain 6 M guanidinium isothiocyanate, 3% 2-mercaptoethanol, 2.5% Triton X-100, 170 ng/µL of proteinase K, and 17 ng/µL of glycogen.

### Sample Collection

Saliva and gargle samples were collected from 23 adult volunteers, including two volunteers who tested SARS-CoV-2 positive using the clinical NPS RT-qPCR test. Multiple samples from the two SARS-CoV-2 positive volunteers, as well as two SARS-CoV-2 negative volunteers who resided in the same household as a SARS-CoV-2 positive volunteer, were collected daily for over four weeks. The first SARS-CoV-2 positive volunteer collected 5 mL of saliva in sterile 50 mL conical tubes (RNase and DNase free; Corning, Corning, NY, USA) during the first week and then collected only morning gargle samples in the following three weeks. During the first week, saliva samples were collected first thing in the morning, at noon, evening, and at night. The second SARS-CoV-2 positive volunteer collected gargle samples first thing in the morning, at noon, evening, and at night during the first week and subsequently collected a single morning gargle sample every day for three weeks. Saliva was produced by actively pooling and spitting into the sterile collection containers.

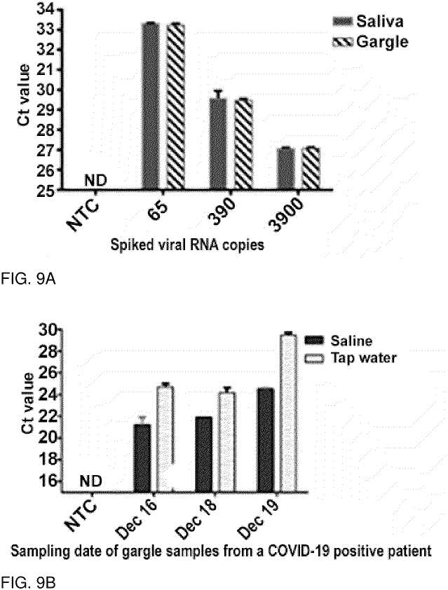

An isotonic saline solution was used for the gargles as saline solution is currently recommended by several studies for mouth rinse/gargle.^{9,} ¹⁸ We also found that SARS-CoV-2 RNA was more stable in saline gargles than in tap water gargles (FIG. 9). One packet of NeilMed Sinus Rinse (NeilMed Pharmaceuticals, Santa Rosa, CA, USA) was dissolved in 480 mL of store-bought bottled spring water (Nestlé, PureLife, Canada). Five milliliters of the saline solution were poured into a sterile 50 mL conical tube (RNase and DNase free; Corning) and poured into the mouth. Volunteers were instructed to gargle and swish for 30 s before spitting back into the same 50 mL conical tube. Then 200 µL of saliva or gargle samples were mixed with 600 µL of VIP buffer. The saliva and gargle samples treated with the VIP buffer were stored at -20° C. until time of analysis. All samples were voluntary, and consent was given for testing. Ethics approval was obtained from the University of Alberta’s Research Ethics Board.

### Pooled Gargle Samples From Multiple Volunteers

Gargle samples from 10 adult volunteers who were previously confirmed negative for SARS-CoV-2 were combined to form a pooled negative gargle sample. This pooled negative gargle sample was used to dilute SARS-CoV-2 positive gargle samples by 8, 16, and 32 times, to simulate a mass population testing scenario where a single positive is present in a pool of 8, 16, and 32 samples, respectively. Three SARS-CoV-2 positive samples were tested for this purpose. The concentrations of SARS-CoV-2 RNA in these positive samples before dilution ranged from 120 copies/200 µL gargle (Ct value 32.2) to 1020 copies/200 µL gargle (Ct value 29.2). The pooled samples of each dilution factor were prepared in triplicate.

### Concentration of RNA on Magnetic Beads

Five hundred microliters of SPRIselect beads (average diameter 1 µm. Beckman Coulter B23317) were washed with RNase-free water three times and resuspended in 10 mL of bead-binding buffer (20 mM Tris-HCl pH 8.0, 2 M NaCl, 36 % PEG-8000, and 2 mM EDTA). This magnetic beads suspension was stored at 4° C. (stable for at least one month). Saliva or gargle samples treated with VIP buffer on-site or mixtures freshly prepared by mixing 200 µL of saliva or gargle samples with 600 µL of VIP buffer in the lab were heated at 55° C. for 10 min and then centrifuged at 13000 g for 5 min. Thereafter, the supernatant was transferred into a new tube and then 400 µL of the magnetic beads suspension and 200 µL of pure ethanol were added into this tube and vortexed for 10 s followed by shaking at room temperature for 10 min. The beads were then collected by centrifugation at 13000 g for 2 min, followed by standing the tubes upright on a magnetic stand to collect the magnetic beads. The resulting beads were washed twice with 0.8 mL of 75% ethanol, followed by air drying for 5 min, and were resuspended in 13.5 µL of RNase-free water containing 200 ng/µL of Proteinase K inhibitor.

### One-step RT-qPCR Analysis of Concentrated Viral RNA Directly on Magnetic Beads

The TaqPath™ 1-Step RT-qPCR Master Mix (ThermoFisher Scientific) and CDC N1 primer-probe from the 2019-nCoV RUO kit (IDT) were used according to the manufacturer’s instructions. The RT-qPCR final reaction volume of 20 µL contained 13.5 µL resuspended magnetic beads solution obtained directly from the concentration step, 5 µL TaqPath 1-Step RT-qPCR Master Mix, and 1.5 µL primer-probe mix (300 nM each primer, 200 nM probe) for the N1 gene segment. The RT-qPCR thermal cycling steps were: 25° C. for 2 min, 50° C. for 15 min, 95° C. for 2 min, and 45 cycles of 95° C. for 3 s and 56° C. for 30 s. RT-qPCR was performed on a QuantStudio™ 3 Real Time PCR System (Applied Biosystems, Thermo Fisher Scientific) using the QuantStudio™ Design & Analysis Software v1.5.1.

### Results and Discussion

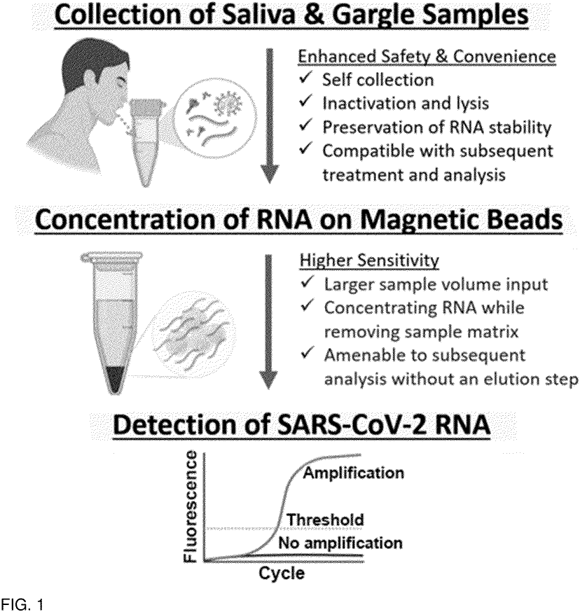

Our method for the detection of SARS-CoV-2 RNA in gargle and saliva samples involves three steps (FIG. 1): (1) self-collection of gargle and/or saliva samples, integrating simultaneous inactivation of viruses with preservation of the viral RNA in the sample solution; (2) capture and concentration of RNA from the collected gargle or saliva sample solution onto magnetic beads; and (3) detection of specific genes of the concentrated SARS-CoV-2 RNA by RT-qPCR without the need for an elution step. During the development of our integrated method, we optimized the concentrations of each reagent in the sample buffer solution to achieve the multiple functions of viral inactivation, RNA release, and preservation of the viral RNA in saliva and gargle samples. We selected appropriate magnetic beads and optimized the conditions for efficient concentration of the viral RNA. In selecting the materials and conditions for the overall assay, we considered and ensured the compatibility of the reagents and processes involved in all three steps for their successful integration into a sensitive VIP-Mag-RT-qPCR assay.

### Development of a Viral Inactivation and RNA Preservation (VIP) Buffer For the Self-Collection of Gargle and Saliva Samples.

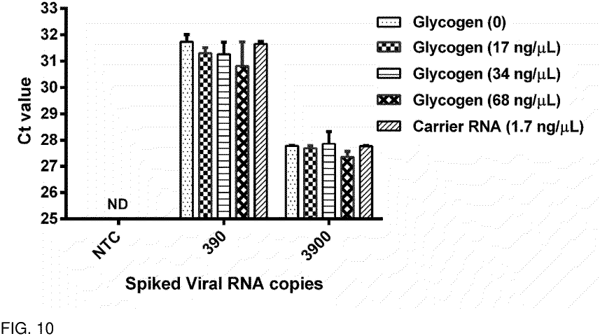

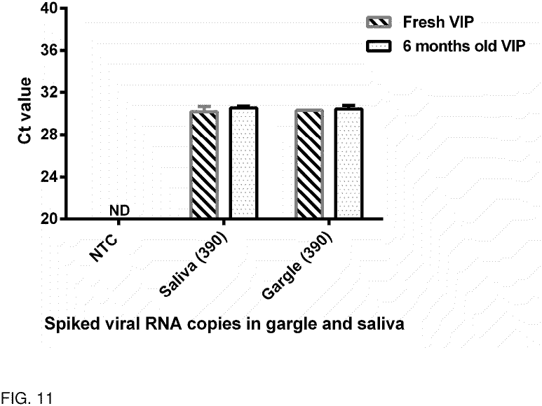

Gargle and saliva samples are heterogeneous and viscous, often containing food remnants and proteins, including RNase enzymes that can digest free RNA. We focused on developing a viral inactivation and RNA preservation (VIP) buffer capable of: (1) inactivating SARS-CoV-2 virus, (2) digesting/dissolving viscous materials and denaturing proteins including RNases, and (3) maintaining the integrity of RNA in the sample. Previous research has shown that surfactants, such as Triton X-100 (2.5-10%), were able to destroy the envelopes of virions and that the addition of Triton X-100 into guanidinium solution enhanced the inactivation of SARS and SARS-CoV-2 viruses.^{7,} ^{20,} ²¹ Two studies also reported that surfactants and guanidinium, such as those in the commercially available (QIAgen) RNA extraction buffers ATL (containing 1%-10% SDS), VXL (containing 30%-50% GuHCl and 2.5%-10% Triton X-100) and buffer RLT (containing guanidinium isothiocyanate),^{21,} ²² were able to inactivate SARS-CoV-2 at a viral load as high as 10⁶ TCID₅₀/mL (median tissue culture infectious dose per milliliter). However, these studies did not aim at preserving or stabilizing the released RNA from the inactivated viruses. Thus, we focused on developing a new formulation that achieves simultaneous viral inactivation and RNA preservation. It is critical that substances (reagents) in the VIP formulation must not affect the subsequent process of detection. To formulate the final VIP buffer, we systematically optimized the composition and concentrations of reagents. We used surfactants and guanidinium to inactivate the virus. We added 2-mercaptoethanol (2-ME) to reduce disulfide bonds and denature proteins such as RNase enzymes that would otherwise degrade RNA. We also included Proteinase K in our formulated VIP buffer to further digest RNases and viscous materials in gargle and saliva samples. We added glycogen as an RNA carrier to enhance the recovery of low concentrations of RNA from samples (FIG. 10).^{23, 24} With a series of optimization experiments, we designed the VIP buffer for gargle and saliva samples to consist of 6 M guanidinium, 3% 2-ME, 2.5% Triton X-100, 170 ng/µL proteinase K, and 17 ng/µL glycogen. We found that 600 µL of this buffer was suitable for mixing with 200 µL of gargle or saliva samples. This VIP buffer is stable for at least 6 months at room temperature (FIG. 11).

### Concentration of Viral RNA Onto Magnetic Beads.

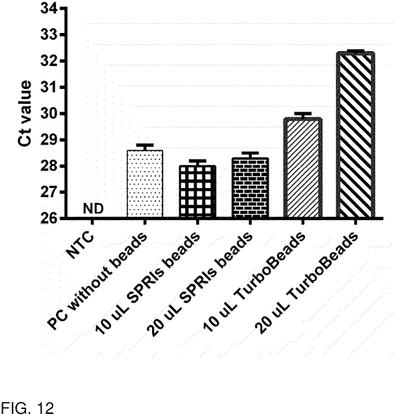

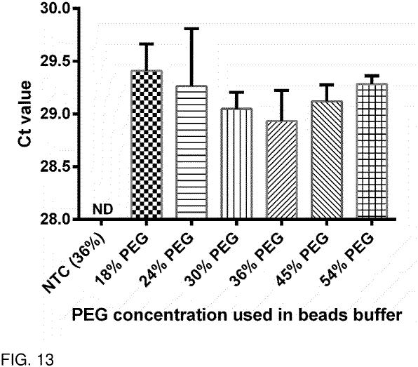

Previous analyses indicated that concentrations of viral RNA in gargle and saliva samples were lower than those in the NPS samples; consequently, the false negative rate was higher for gargle and saliva than for NPS tests.¹⁶ To overcome this problem, we concentrated viral RNA on magnetic beads for the subsequent RT-qPCR analysis, to achieve two benefits: (1) increase the sensitivity of the assay by concentrating the target viral RNA, and (2) increase the specificity by removing sample matrix materials that could interfere with the RT-qPCR analysis. After preliminary tests of various magnetic beads, we chose to use SPRIselect magnetic beads to capture nucleic acids including the viral RNA. We tested RT-qPCR analysis of the viral RNA in the presence of magnetic beads (FIG. 12) with the final goal of conducting RT-qPCR analysis directly on the magnetic beads without the need for an RNA elution step. We found that the threshold cycle (Ct) values from the RT-qPCR analysis of 2000 copies of the SARS-CoV-2 RNA were similar in the presence and absence of the SPRIselect magnetic beads (FIG. 13). These results indicate that the SPRIselect beads themselves did not inhibit RT-qPCR. We then optimized the concentration of PEG-8000 in the binding buffer for efficient capture of the SARS-CoV-2 RNA on the magnetic beads (FIG. 13). The presence of PEG [H-(O-CH2-CH2)n-OH] and Na⁺ ions in the binding solution is important for concentration of viral RNA onto the SPRIselect beads, because PEG removes water surrounding the RNA and Na⁺ ions shield the negative phosphate backbones, making RNA readily adsorbed onto the beads.²⁵ We found that the optimal buffer consisted of 20 mM Tris-HCl at pH 8.0, 2 M NaCl, 2 mM EDTA, and 36% PEG-8000.

### RT-qPCR Analysis of Viral RNA Directly From Magnetic Beads.

To simplify the assay, we conducted RT-qPCR analysis of the concentrated viral RNA directly on the magnetic beads, achieving two main benefits: shortening the sample preparation time and minimizing the loss of the target viral RNA. To prevent a potential problem of Proteinase K unintentionally being adsorbed on the magnetic beads, we included a Proteinase K inhibitor (tetrapeptidyl chloromethyl ketone) in the RT-qPCR reaction solution. Therefore, the magnetic beads that captured viral RNA from the gargle and saliva samples could be directly analyzed without the need for a traditional RNA elution step and without the negative impact of RT-qPCR inhibition.

### Overall VIP-Mag-RT-qPCR Method for the Detection of SARS-CoV-2 in Gargle and Saliva.

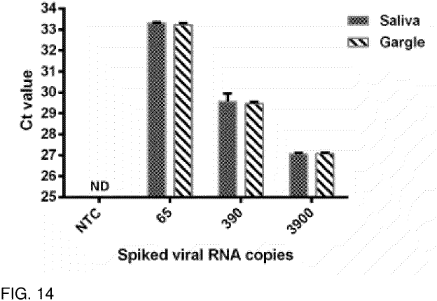

We tested our VIP-Mag-RT-qPCR method on pooled gargle samples and pooled saliva samples from ten SARS-CoV-2 negative volunteers, as well on as the pooled samples supplemented with SARS-CoV-2 RNA at three concentrations, 65, 390, and 3900 copies per 200 µL sample. Our results (FIG. 14) show positive detection of the N gene of SARS-CoV-2 in both the gargle and saliva samples containing as few as 65 copies of the viral RNA. For all three pairs of gargle and saliva samples containing varying concentrations of the viral RNA, the Ct values were consistent and as expected. These results demonstrate that the overall integrated VIP-Mag-RT-qPCR method is suitable for the determination of SARS-CoV-2 RNA in both gargle and saliva samples.

### Recovery of Viral RNA From Gargle and Saliva Samples.

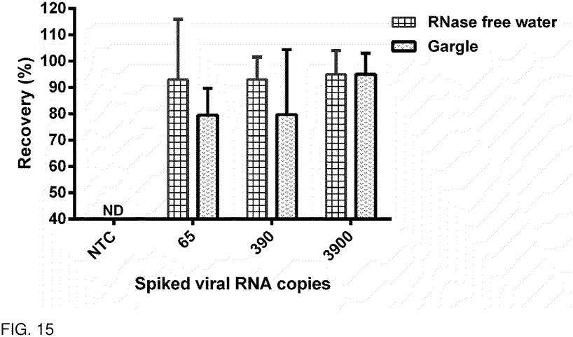

To test the overall recovery of the viral RNA, we prepared a pooled gargle sample from ten SARS-CoV-2 negative volunteers and spiked each 200 µL pooled sample with 65, 390, or 3900 copies of SARS-CoV-2 RNA. We determined that the overall recovery of the added SARS-CoV-2 RNA was 80-95% (FIG. 15). For comparison, we also added 65, 390, or 3900 copies of SARS-CoV-2 RNA into 200 µL of RNase-free water and determined that the overall recovery of the SARS-CoV-2 RNA was 93-95%. The recovery of 3900 copies of SARS-CoV-2 RNA from both the pooled gargle sample and the RNase-free water was identical (95%). These results show acceptable overall recovery (80-95%) over a wide range of SARS-CoV-2 RNA concentrations (from 65 to 3900 copies per 200 µL) from both gargle and water samples.

### Stability and Preservation of Viral RNA in the VIP Buffer.

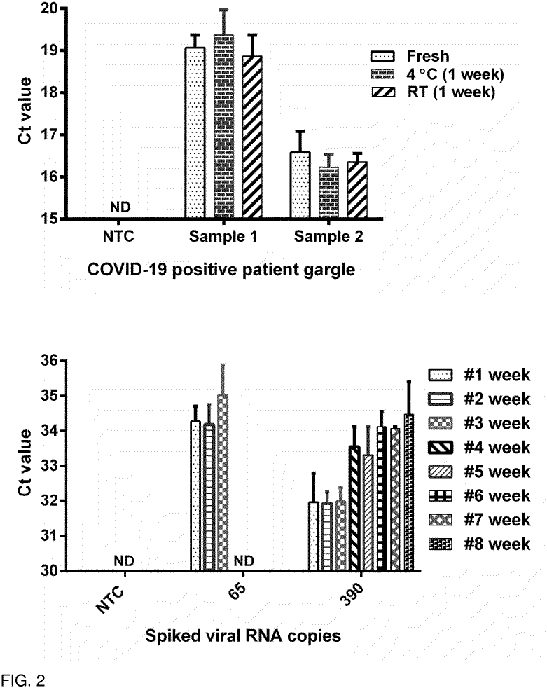

We tested the stability of SARS-CoV-2 RNA in gargle samples placed in the VIP buffer at room temperature or 4° C. for up to eight weeks (FIG. 2). We treated two gargle samples collected from a SARS-CoV-2 NPS-positive patient volunteer on different days. We mixed 200 µL of each sample with 600 µL of the VIP buffer, generating nine replicate aliquots. We analyzed the first three aliquots on the sample collection day and stored the remaining six replicate aliquots for one week, three of which at 4° C. and the other three at room temperature. RT-qPCR analysis indicates that Ct values are similar for all three sets of triplicates (FIG. 2A). Therefore, SARS-CoV-2 RNA is stable at room temperature or 4° C. for at least one week in gargle samples mixed with the VIP buffer. We also tested the stability of 65 and 390 copies of SARS-CoV-2 RNA added into gargle samples (FIG. 2B). The gargle samples containing 65 copies of viral RNA and the VIP buffer remain positive after storage for up to three weeks at room temperature (FIG. 2B). The samples containing 390 copies of viral RNA and the VIP buffer remain positive after storage for eight weeks at room temperature. There is no change in the Ct values between the samples stored for one week and two weeks. These results suggest that the VIP buffer is suitable for preserving samples for two weeks, which is particularly useful for testing in remote areas where the collected samples cannot be routinely delivered to a clinical laboratory but can be delivered within 2-3 weeks.

### Sensitivity and Repeatability of the VIP-Mag-RT-qPCR Method for Detection of SARS-CoV-2 in Gargle Samples.

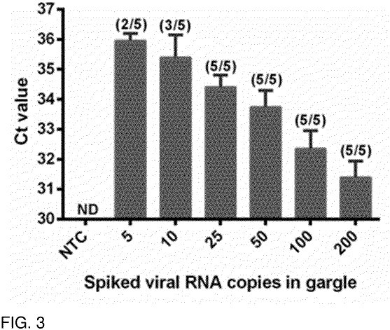

We tested the sensitivity of the VIP-Mag-RT-qPCR method for gargle samples prepared with known amounts of viral RNA. We added 5-200 copies of viral RNA into 200 µL of pooled negative gargle samples. Each concentration condition was prepared in five replicates. We found that all five replicates (100%) of the gargle sample tested positive when the number of viral RNA copies was ≥25 (FIG. 3). Ten viral RNA copies were detected in three replicates and five viral RNA copies were detected in two replicates. These results indicate that the integrated VIP-Mag-RT-qPCR method is very sensitive and capable of testing as low as 25 copies/200 µL of gargle samples.

We repeatedly tested samples containing very low concentrations of viral RNA on multiple days. We prepared 30 replicates of gargle samples containing 100 copies of viral RNA. We processed and tested these samples on three consecutive days (10 samples/day). The intra-day coefficient variation (CV) is 1.7%, and the inter-day variation is 2.1%, indicating that the method yields consistent results even with only 100 copies of viral RNA present in 200 µL of gargle.

### Comparison of the VIP-Mag Method With the CDC-Recommended Commercial Kit

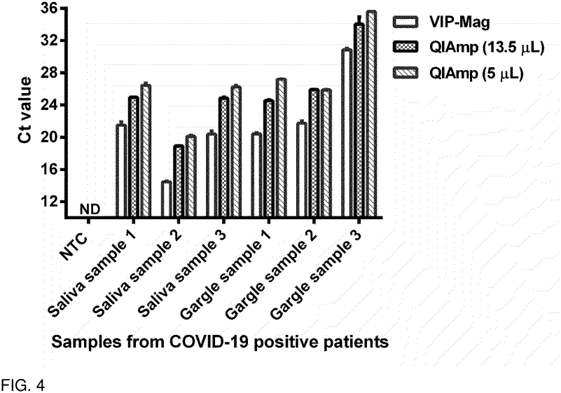

We compared our VIP-Mag method with the commercial QIAamp Viral RNA Mini Kit recommended by the CDC for the detection of SARS-CoV-2. The QIAamp Viral RNA Mini Kit is widely used for extracting SARS-CoV-2 RNA from clinical NPS samples. We concurrently conducted triplicate analyses of three saliva samples and three gargle samples from SARS-CoV-2 patients. Compared to our VIP-Mag method, the method using the recommended QIAamp Viral RNA Mini Kit consistently required more PCR cycles to achieve positive detection of SARS-CoV-2 RNA (FIG. 4). Although a similar volume (140-200 µL) of gargle and saliva samples was used with both methods, the QIAamp Viral RNA Mini Kit protocol required an elution step and only a fraction (5 µL recommended or 13.5 µL maximum) of 60 µL eluted RNA could be analyzed. In contrast, the VIP-Mag method is able to concentrate RNA from samples and directly analyze RNA on magnetic beads without requiring an elution step. The difference in Ct values between these two methods is 3.2-6.8 cycles (FIG. 4), corresponding to 9-111 times in analytical sensitivity. This sensitivity improvement of the VIP-Mag method is attributed to a combination of the efficient concentration of RNA on magnetic beads and elimination of PCR inhibitors from the samples. Incomplete removal of PCR inhibitors from gargle sample #2 by the QIAamp Viral RNA Mini Kit could be a reason for no difference in the Ct value (25.9) when the RNA input volume was increased from 5 µL to 13.5 µL (FIG. 4). In principle, an increase of the RNA input volume would reduce Ct cycles needed for positive detection. However, if PCR inhibitors were not removed, the increase of input sample volume would also increase the amount of PCR inhibitors affecting amplification. Thus, researchers using commercial kits to extract RNA have attempted to dilute saliva samples to lower the inhibitor concentration; but dilution of samples decreases detection sensitivity.^{9,17} Efficient removal of PCR inhibitors overcame this problem.

### Detection of SARS-CoV-2 in Patients’ Gargle and Saliva Samples Collected from the Onset of Clinical Symptom through to Recovery

We monitored SARS-CoV-2 levels in gargle and saliva samples from two NPS-confirmed SARS-CoV-2 positive patients for about a month, covering the period of clinical symptoms and recovery. In parallel, we also tested samples from 21 volunteers, including two people who were NPS-confirmed SARS-CoV-2 negative and lived in the same house with a NPS-confirmed SARS-CoV-2 positive patient. In total, 123 samples were collected from two positive patients and 56 samples were collected from two negative volunteers from the same household.

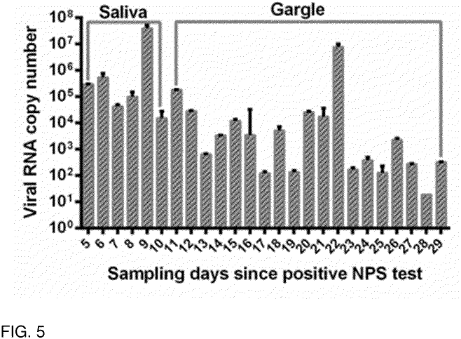

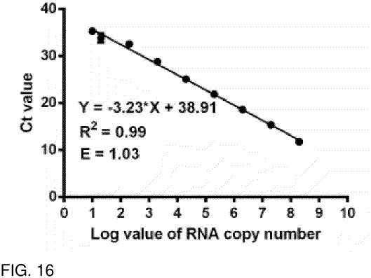

The first patient volunteer had initial symptoms of a mild cough and fever before NPS testing positive for SARS-CoV-2. Self-collection of saliva and gargle samples started on the fifth day after the positive NPS test, and continued for 25 days. We analyzed the gargle and saliva samples and estimated the viral RNA levels by converting Ct values to viral RNA copies based on the RT-qPCR standard curve of the N1 gene segment (FIG. 16). As shown in FIG. 5, as high as 10⁷ copies of the viral RNA were present in 200 µL of saliva or gargle samples. All the samples collected from this patient throughout 25 days had detectable SARS-CoV-2 RNA. Parallel analyses of saliva and gargle samples collected on the same day from two COVID-negative volunteers who lived in the same house as the patient showed no detectable SARS-CoV-2 RNA. These results confirmed that there was no false-positive in the detection of SARS-CoV-2 RNA using our method.

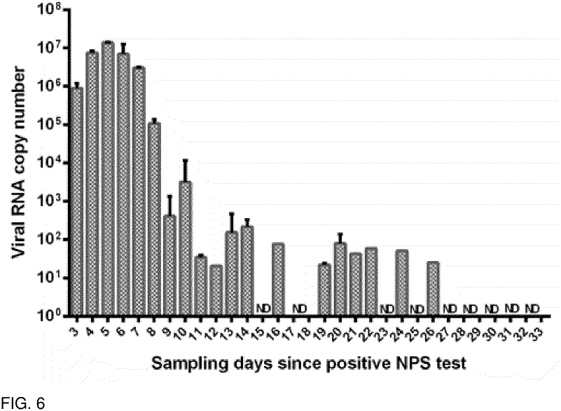

The second SARS-CoV-2 positive patient volunteer started to have a fever two days before positive NPS test. The patient provided morning gargle samples daily from the third day to thirty-third day since positive test, and collected four gargle samples daily from the fourth day to the eleventh day. We tested all the samples and the data from all the morning gargles are shown in FIG. 6. These results show a large variation of the viral RNA levels in gargle over a period of month. While some samples had 10⁷ copies of viral RNA in 200 µL gargle, a number of samples had viral RNA concentrations below 100 copies in 200 µL gargle. To confirm positive detections of these low concentrations of viral RNA, we concurrently analyzed gargle samples collected from 19 volunteers who were COVID-negative. All the samples from the COVID-negative volunteers had no detectable SARS-CoV-2 RNA.

Interestingly, both patients had detectable SARS-CoV-2 RNA in their gargle samples for more than two weeks. A systematic review of 28 studies²⁶ has shown that the median duration of SARS-CoV-2 RNA shedding from respiratory sources was 18.4 days. The duration of SARS-COV-2 RNA shedding has high heterogeneity and viral RNA was detected up to 92 days after symptom onset.²⁶

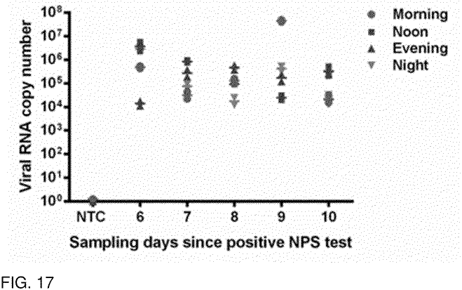

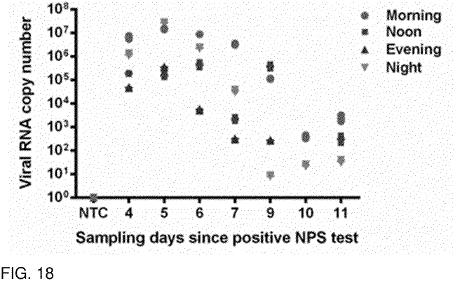

We also investigated whether there was any difference in viral RNA levels depending on the time of collection throughout the day. All saliva samples collected in the first five days from patient one had viral RNA concentrations higher than 10⁴ copies/200 µL (FIG. 5), but showed no temporal pattern (FIG. 17). Four gargle samples (morning, noon, evening, and night) collected consecutively for seven days from patient two showed that the viral RNA concentration was the highest in the morning samples for five of the seven sampling days (FIG. 18). These results indicate consistent detection in all samples throughout the day, although a trend within a day cannot be identified from samples of the two patients.

### Application of the VIP-Mag-RT-qPCR Method to the Analysis of Pooled Samples

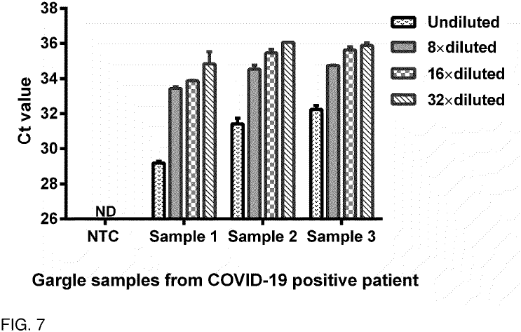

Because our VIP-Mag-RT-qPCR method can detect as few as 25 copies of viral RNA/200µL gargle, such high sensitivity makes it possible to analyze pooled samples collected from multiple individuals. When the positivity rate is low, analysis of pooled samples could save resources for mass population surveillance.²⁷⁻³⁰ We chose negative gargle samples from ten healthy volunteers and three SARS-CoV-2 positive samples containing approximately 120 copies (Ct value 32.2), 210 copies (Ct value 31.4), and 1020 copies (Ct value 29.2) of SARS-CoV-2 RNA per 200 µL gargle, respectively. To simulate a pool of one positive in 8, 16, or 32 samples, we diluted a positive sample with the pooled negative gargle sample by 8, 16, and 32 times. Our VIP-Mag-RT-qPCR analyses of these diluted samples show consistently positive detections (FIG. 7). Thus, a single positive gargle sample pooled with 7, 15, or 31 other negative gargle samples can be detected positive despite the dilution of the positive sample. These results demonstrate successful application of our VIP-Mag-RT-qPCR method to the analysis of pooled gargle samples and suggest its potential for the surveillance of COVID-19 in a large population.

### Conclusions

We developed a VIP-Mag-RT-qPCR method which includes an inexpensive solution enabling on-site sample self-collection with integrated virus inactivation and RNA preservation, magnetic beads-mediated RNA concentration, and direct analysis of the viral RNA concentrated on the magnetic beads without the need for elution. This method simplifies procedures of collection and treatment of saliva and gargle samples for detection of SARS-CoV-2. The VIP-Mag-RT-qPCR method is able to detect as few as 25 copies of viral RNA in 200 µL of samples, improving the detection sensitivity by 9 to 111 times over the QIAamp Viral RNA Mini Kit recommended by the CDC. The improved method has sufficient sensitivity to detect diluted level of SARS-CoV-2 in pooled samples for potential population surveillance. The VIP buffer can be used for self-collection of samples and on-site inactivation, minimizing the risks associated with viral spreading and transmission to healthcare and laboratory personnel during collection and analysis of clinical samples. Saliva and gargle samples are complementary to nasopharyngeal swabs, providing an alternative for repeated sampling, population surveillance, and point-of-care testing of SARS-CoV-2. This research addresses a need for molecular diagnosis of COVID-19.³¹ The general strategy of viral inactivation and nucleic acid preservation could potentially be applied to the detection of other enveloped viruses.

### Associated Content

### Supporting Information

Additional experimental details and methods, including optimization experiments and comparison of various methods.

### References

(1) Azzi, L.; Maurino, V.; Baj, A.; Dani, M.; d′Aiuto, A.; Fasano, M.; Lualdi, M.; Sessa, F.; Alberio, T. Diagnostic salivary tests for SARS-CoV-2. J. Dent. Res. 2021, 100(2), 115-123. DOI: 10.1177/0022034520969670
(2) Wiersinga, J. W.; Rhodes, A.; Cheng, A. C.; Peacock, S. J.; Prescott, H. C. Pathophysiology, transmission, diagnosis, and treatment of coronavirus disease 2019 (COVID-19). JAMA. 2020, 324(8),782-793. DOI: 10.1001/jama.2020.12839
(3) Wölfel, R.; Corman, V. M.; Guggemos, W.; Seilmaier, M.; Zange, S.; Miller, M. A.; Niemeyer, D.; Jones, T. C.; Vollmar, P.; Rothe, C.; Hoelscher, M; Bleicker, T.; Brunink, S.; Schneider, J.; Ehmann, R.; Zwirglmaier, K.; Drosten, C.; Wendtner, C. Virological assessment of hospitalized patients with COVID-2019. Nature 2020, 581(7809), 465-469. DOI: 10.1038/s41586-020-2196-x
(4) Zou, L.; Ruan, F.; Huang, M.; Liang, L.; Huang, H.; Hong, Z.; Yu, J.; Kang, M.; Song, Y.; Xia, J.; Guo, Q. SARS-CoV-2 viral load in upper respiratory specimens of infected patients. N. Engl. J. Med. 2020, 382(12), 1177-1179. DOI: 10.1056/NEJMc2001737
(5) Kucirka, L. M.; Lauer, S. A.; Laeyendecker, O.; Boon, D.; Lessler, J. Variation in false-negative rate of reverse transcriptase polymerase chain reaction-based SARS-CoV-2 tests by time since exposure. Ann. Intern. Med. 2020, 173(4), 262-267. DOI: 10.7326/M20-1495
(6) Khurshid, Z.; Zohaib, S.; Najeeb, S.; Zafar, M. S.; Slowey, P. D.; Almas, K. Human saliva collection devices for proteomics: an update. Int. J. Mol. Sci. 2016, 17(6): 846. DOI: 10.3390/ijms17060846
(7) Wang, W. K.; Chen, S. Y.; Liu, I. J.; Chen, Y. C.; Chen, H. L.; Yang, C. F.; Chen, P. J.; Yeh, S. H.; Kao, C. L.; Huang, L. M.; Hsueh, P. R. Detection of SARS-associated coronavirus in throat wash and saliva in early diagnosis. Emerg. Infect. Dis. 2004, 10(7), 1213-1219. DOI: 10.3201/eid1007.031113
(8) Wyllie, A. L.; Fournier, J.; Casanovas-Massana, A.; Campbell, M.; Tokuyama, M.; Vijayakumar, P.; Warren, J. L.; Geng, B.; Muenker, M.C.; Moore, A. J.; Vogels, C. B. Saliva or nasopharyngeal swab specimens for detection of SARS-CoV-2. N. Engl. J. Med. 2020, 383, 1283-1286. DOI: 10.1056/NEJMc2016359
(9) Goldfarb, D. M.; Tilley, P.; Al-Rawahi, G. N.; Srigley, J. A.; Ford, G.; Pedersen, H.; Pabbi, A.; Hannam-Clark, S.; Charles, M.; Dittrick, M.; Gadkar, V. J. Self-collected saline gargle samples as an alternative to health care worker-collected nasopharyngeal swabs for COVID-19 diagnosis in outpatients. J. Clin. Microbiol. 2021, 59(4), e02427-20. DOI: 10.1128/JCM.02427-20
(10) Kojima, N.; Turner, F.; Slepnev, V.; Bacelar, A.; Deming, L.; Kodeboyina, S.; Klausner, J. D. Self-collected oral fluid and nasal swabs demonstrate comparable sensitivity to clinician collected nasopharyngeal swabs for coronavirus disease 2019 detection. Clin. Infect. Dis. 2021, 73(9), e3106-e3109. DOI: 10.1093/cid/ciaa1589
(11) Vogels, C. B.; Watkins, A.E.; Harden, C. A.; Brackney, D. E.; Shafer, J.; Wang, J.; Caraballo, C.; Kalinich, C. C.; Ott, I. M.; Fauver, J. R.; Kudo, E.; Piewan, L.; Venkataram, A.; Tokuyama, M.; Moore, A.J.; Muenker, M.C.; Casanovas-Massana, A.; Fournier, J.; Bermejo, S.; Campbell, M.; Datta, R.; Nelson, A.; Cruz, C.S.D.; Farhadian, S.F.; Ko, A.I.; Iwasaki, A.; Krumholz, H.M.; Matheus, J.D.; Hui, P.; Liu, C.; Sikka, R.; Wyllie, A. L.; Grubaugh, N. D. SalivaDirect: A simplified and flexible platform to enhance SARS-CoV-2 testing capacity. Med 2021, 2(3), 263-280. DOI: 10.1016/j.medj.2020.12.010
(12) Hall, E. W.; Luisi, N.; Zlotorzynska, M.; Wilde, G.; Sullivan, P.; Sanchez, T.; Bradley, H.; Siegler, A. J. Willingness to use home collection methods to provide specimens for SARS-CoV-2/COVID-19 research: survey study. J. Med. Internet Res. 2020, 22(9), e19471. DOI: 10.2196/19471
(13) Moreno-Contreras, J.; Espinoza, M. A.; Sandoval-Jaime, C.; Cantú-Cuevas, M. A.; Barón-Olivares, H.; Ortiz-Orozco, O. D.; Munoz-Rangel, A. V.; Hernández-de la Cruz, M.; Eroza-Osorio, C. M.; Arias, C. F.; López, S. Saliva sampling and its direct lysis, an excellent option to increase the number of SARS-CoV-2 diagnostic tests in settings with supply shortages. J. Clin. Microbiol. 2020, 58(10), e01659-20. DOI: 10.1128/JCM.01659-20
(14) Ott, I. M.; Strine, M. S.; Watkins, A. E.; Boot, M.; Kalinich, C. C.; Harden, C. A.; Vogels, C. B.; Casanovas-Massana, A.; Moore, A. J.; Muenker, M. C.; Nakahata, M. Stability of SARS-CoV-2 RNA in nonsupplemented saliva. Emerg. Infect. Dis. 2021, 27(4), 1146-1150. DOI: 10.3201/eid2704.204199
(15) Griesemer, S. B.; Van Slyke, G.; Ehrbar, D.; Strle, K.; Yildirim, T.; Centurioni, D. A.; Walsh, A. C.; Chang, A. K.; Waxman, M. J.; St. George, K. Evaluation of specimen types and saliva stabilization solutions for SARS-CoV-2 testing. J. Clin. Microbiol. 2021, 59(5), e01418-20. DOI: 10.1128/JCM.01418-20
(16) Lee, R. A.; Herigon, J. C.; Benedetti, A.; Pollock, N. R.; Denkinger, C. M. Performance of saliva, oropharyngeal swabs, and nasal swabs for SARS-CoV-2 molecular detection: A systematic review and meta-analysis. J. Clin. Microbiol. 2021, 59(5), e02881-20. DOI: 10.1128/JCM.02881-20
(17) Mohammadi, A.; Esmaeilzadeh, E.; Li, Y.; Bosch, R. J.; Li, J. Z. SARS-CoV-2 detection in different respiratory sites: A systematic review and meta-analysis. EBioMedicine 2020, 59, 102903. DOI: 10.1016/j.ebiom.2020.102903
(18) Kandel, C. E.; Young, M.; Serbanescu, M. A.; Powis, J. E.; Bulir, D.; Callahan, J.; Katz, K.; McCready, J.; Racher, H.; Sheldrake, E.; Quon, D.; Vojdani, O. K.; McGeer, A.; Goneau, L. W.; Vermeiren, C. Detection of severe acute respiratory coronavirus virus 2 (SARS-CoV-2) in outpatients: A multicenter comparison of self-collected saline gargle, oral swab, and combined oral-anterior nasal swab to a provider collected nasopharyngeal swab. Infect. Control Hosp. Epidemiol. 2021, 42(11), 1340-1344. DOI: 10.1017/ice.2021.2
(19) Joung, J.; Ladha, A.; Saito, M.; Kim, N. G.; Woolley, A. E.; Segel, M.; Barretto, R. P.; Ranu, A.; Macrae, R. K.; Faure, G.; Ioannidi, E. I. Detection of SARS-CoV-2 with SHERLOCK one-pot testing. N. Engl. J. Med. 2020, 383(15), 1492-1494. DOI: 10.1056/NEJMc2026172
(20) Patterson, E. I.; Prince, T.; Anderson, E. R.; Casas-Sanchez, A.; Smith, S. L.; Cansado-Utrilla, C.; Solomon, T.; Griffiths, M. J.; Acosta-Serrano, Á.; Turtle, L.; Hughes, G. L. Methods of inactivation of SARS-CoV-2 for downstream biological assays. J. Infect. Dis. 2020, 222(9), 1462-1467. DOI: 10.1093/infdis/jiaa507
(21) Welch, S. R.; Davies, K. A.; Buczkowski, H.; Hettiarachchi, N.; Green, N.; Arnold, U.; Jones, M.; Hannah, M. J.; Evans, R.; Burton, C.; Burton, J. E; Guiver, M.; Cane, P. A.; Woodford, N.; Bruce, C. B.; Roberts, A. D. G.; Killip, M. J. Analysis of inactivation of SARS-CoV-2 by specimen transport media, nucleic acid extraction reagents, detergents, and fixatives. J. Clin. Microbiol. 2020, 58(11), e01713-20. DOI: 10.1128/JCM.01713-20
(22) Pastorino, B.; Touret, F.; Gilles, M.; Luciani, L.; de Lamballerie, X.; Charrel, R. N. Evaluation of chemical protocols for inactivating SARS-CoV-2 infectious samples. Viruses 2020, 8, 12(6):624. DOI: 10.3390/v12060624
(23) Andreasen, D.; Fog, J. U.; Biggs, W.; Salomon, J.; Dahslveen, I. K.; Baker, A.; Mouritzen, P. Improved microRNA quantification in total RNA from clinical samples. Methods 2010, 50(4), S6-S9. DOI: 10.1016/j.ymeth.2010.01.006
(24) Ramón-Núñez, L. A.; Martos, L.; Fernández-Pardo, Á.; Oto, J.; Medina, P.; España, F.; Navarro, S. Comparison of protocols and RNA carriers for plasma miRNA isolation. Unraveling RNA carrier influence on miRNA isolation. PLoS One 2017, 12(10), e0187005. DOI: 10.1371/journal.pone.0187005
(25) Bloomfield, V. A. DNA condensation by multivalent cations. Biopolymers 1997, 44(3), 269-282. DOI: 10.1002/(SICI)1097-0282(1997)44:3<269::AID-BIP6>3.0.CO;2-T
(26) Fontana, L.; Villamagna, A. H.; Sikka, M. K.; McGregor, J. C. Understanding viral shedding of SARS-CoV-2: review of current literature. Infec. Control Hosp. Epidemiol. 2020, 42(6), 659-668. DOI: 10.1017/ice.2020.1273
(27) Schulte, P. A.; Weissman, D. N.; Luckhaupt, S. E.; de Perio, M. A.; Beezhold, D.; Piacentino, J. D.; Radonovich, L. J. Jr; Hearl, F. J.; Howard, J. Considerations for pooled testing of employees for SARS-CoV-2. J. Occupat. Environ. Med. 2021, 63(1), 1-9. DOI: 10.1097/JOM.0000000000002049
(28) Cleary, B.; Hay, J. A.; Blumenstiel, B.; Harden, M.; Cipicchio, M.; Bezney, J.; Simonton, B.; Hong, D.; Senghore, M.; Sesay, A. K.; Gabriel, S.; Regev, A.; Mina, M. J. Using viral load and epidemic dynamics to optimize pooled testing in resource-constrained settings. Sci. Translation. Med. 2021, 13 (589), eabf1568. DOI: 10.1126/scitranslmed.abf1568
(29) Grobe, N.; Cherif, A.; Wang, X. L.; Dong, Z. J.; Kotanko, P. Sample pooling: burden or solution? Clin. Microbiol. Infect. 2021, 27 (9), 1212-1220. DOI: 10.1016/j.cmi.2021.04.007
(30) Deka, S.; Kalita, D. Effectiveness of sample pooling strategies for SARS-CoV-2 mass screening by RT-PCR: A scoping review. J. Lab. Physicians 2020, 12 (3), 212-218. DOI: 10.1055/s-0040-1721159
(31) Feng, W.; Newbigging, A. M.; Le, C.; Pang, B.; Peng, H.; Cao, Y.; Wu, J.; Abbas, G.; Song, J.; Wang, D.-B.; Cui, M.; Tao, J.; Tyrrell, D. L.; Zhang, X.-E.; Zhang, H.; Le, X. C. Molecular diagnosis of COVID-19: Challenges and research needs. Anal. Chem. 2020, 92 (15), 10196-10209. DOI: 10.1021/acs.analchem.0c02060.

Table 1. Comparison of reported methods on inactivation, RNA release and extraction for the detection of SARS-CoV-2 in saliva and gargle samples. A single checkmark indicates the study partially addressed a certain aspect. Two checkmarks indicates the study fully addressed a certain aspect.

TABLE 1

| | | | | | |
|---|---|---|---|---|---|
| Study | Sample | Inactivation/Extrac tion | RNA Release & Stability | Detection Method | Sensitivity |
| VIP-Mag Method | BOTH | ✓✓ | ✓✓ | ✓✓ | ✓✓ |
| | Saliva & Gargle | Single Tube Method (sample collection, inactivation, lysis, & maintains RNA stability) Heat at 55° C., 10 min | 1 week at 4° C. & Room Temperature Directly inputted into RT-qPCR without the need for elution/purification | RT-qPCR | 25 RNA copies/ 200 µL of sample |
| Ranoa et al., 2020 | Saliva | ✓ | ✓ | ✓✓ | ✓ |
| | Diluted in TE/TBE buffer to reduce viscosity | Heat at 95° C. for 30 min TBE buffer and Tween 20 | 24 hours at 4° C. (RNA) Directly inputted into RT-qPCR without the need for elution/purification | RT-qPCR | 500-1000 viral particles/mL of saliva |
| Vogels et al., 2020 | Saliva | ✓ | ✓ | ✓✓ | ✓✓ |
| | | Heat at 95° C. for 5 min Proteinase K | 1 week at 30° C., 4° C. & Room Temperature (SARS-CoV-2 in saliva pre-inactivation/extraction) Directly inputted into RT-qPCR without the need for elution/purification | RT-qPCR | 6-12 SARS-CoV-2 copies/µL of saliva |
| Lalli et al., 2021 | Saliva | ✓ | X | ✓✓ | ✓ |
| | Diluted in phosphate buffered saline (PBS) to reduce viscosity | Heat at 65° C. for 15 min, 95° C. for 5 min, and cooled to 4° C. RNAsecure & Proteinase K to each sample | RNA stability not mentioned Directly inputted into RT-qPCR and LAMP without the need for elution/purification | RT-qPCR LAMP | 100 viral genomes/reaction |
| Yang et al., 2021 | Saliva | ✓ | ✓ | ✓✓ | ✓ |
| | | Heat at 95° C. for 10 min Saliva stabilization solution (5 mM TCEP, 2 mM EDTA, 29 mM NaOH, 100 µg/mL Proteinase K) | 4 days at 4° C. (RNA) | LAMP | 200 virions/µL of saliva |
| Tilley et al., 2021 | Gargle | ✓ | X | ✓✓ | X |
| | Saline solution | Heat at 65° C. for 20 min and cool to room temperature for 5 min | RNA stability not mentioned Directly inputted into RT-qPCR without the need for elution/purification | RT-qPCR Single-tube hemi-nested real-time-qPCR (STHN-RT-qPCR) to enhance the overall sensitivity | 97% match to NPS positive samples (viral number or copy number not mentioned) |

### References:

Lalli, M.A.; Langmade, J.S.; Chen, X.; Fronick, C.C.; Sawyer, C.S.; Burcea, L.C.; Wilkinson, M.N.; Fulton, R.S.; Heinz, M.; Buchser, W.J.; Head, R.D.; Mitra, R.D.; Milbrandt, J. Rapid and extraction-free detection of SARS-CoV-2 from saliva by colorimetric reverse-transcription loop-mediated isothermal amplification. Clin. Chem. 2021, 67(2),415-424. DOI: 10.1093/clinchem/hvaa267
Ranoa, D.; Holland, R.; Alnaji, F.G.; Green, K.; Wang, L.; Brooke, C.; Burke, M.; Fan. T.; Hergenrother, P.J. Saliva-based molecular testing for SARS-CoV-2 that bypasses RNA extraction. Biorxiv. 2020. DOI: HYPERLINK “https://d.docs.live.net/26efea6a3c49de6b/Documents/Saliva SARS-COV-2/Manuscript/10.1101/2020.06.18.159434″10.1101/2020.06.18.159434 (accessed 2021 Dec 10)
Tilley, P.; Gadkar, V.; Goldfarb, D.M.; Young, V.; Watson, N.; Al-Rawahi, G.N.; Srigley, J.; Hoang, L.; Lee, T.; Prystajecky, N. Gargle-Direct: Extraction-Free Detection of SARS-CoV-2 using Real-time PCR (RT-qPCR) of Saline Gargle Rinse Samples. medRxiv. 2020. DOI: 10.1101/2020.10.09.20203430 (accessed 2021 Dec 10).
Vogels, C.B.; Brackney, D.; Wang, J.; Kalinich, C.C.; Ott, I.; Kudo, E.; Lu, P.; Venkataraman, A.; Tokuyama, M.; Moore, A.J.; Muenker, M.C.; Casanovas-Massana, A.; Fournier, J.; Bermejo, S.; Campbell, M.; Datta, R.; Nelson, A.; Cruz, C.S.D.; Farhadian, S.F.; Ko, A.I.; Iwasaki, A.; Krumholz, H.M.; Matheus, J.D.; Hui, P.; Liu, C.; Sikka, R.; Wyllie, A.L.; Grubaugh, N.D. SalivaDirect: Simple and sensitive molecular diagnostic test for SARS-CoV-2 surveillance. Med 2021, 2(3), 263-280. DOI: HYPERLINK “https://d.docs.live.net/26efea6a3c49de6b/Documents/Saliva SARS-COV-2/Manuscript/10.1016/j.medj.2020.12.010″10.1016/j.medj.2020.12.010
Yang, Q.; Meyerson, N.R.; Clark, S.K.; Paige, C.L.; Fattor, W.T.; Gilchrist, A.R.; Barbachano-Guerrero, A.; Healy, B.G.; Worden-Sapper, E.R.; Wu, S.S.; Muhlrad, D.; Cecker, C.J.; Saldi, T.K.; Lasda, E.; Gonzales, P.; Fink, M.R.; Tat, K.L.; Hager, C.R.; Davis, J.C.; Ozeroff, C.D.; Brisson, G.R.; McQueen, M.B.; Leinwand, L.A.; Parker, R.; Sawyer, S.L. Saliva TwoStep for rapid detection of asymptomatic SARS-CoV-2 carriers. eLife. 2021, 10: e65113. DOI: 10.7554/eLife.65113

Table 2. A summary of studies on the detection of SARS-CoV-2 in saliva. Inactivation/extraction method, RNA stability, detection method, and sensitivity comparison with nasopharyngeal swabs (NPS) analysis are summarized. The checkmarks indicate that the study addressed a certain aspect.

TABLE 2

| | | | | | |
|---|---|---|---|---|---|
| Study | Sample | Inactivation/Extracti on | RNA Stability | Detection Method | Comparison to NPS positive samples |
| Altawalah et al., 2020 | Saliva | ✓ | X | ✓ | ✓ |
| | Mixed in VTM | MagMax Viral/Pathogen Nucleic Acid Isolation Kit | Not mentioned | RT-qPCR | 91 % match to NPS |
| Aita et al., 2020 | Saliva | X | X | ✓ | ✓ |
| | Salivette® | Not mentioned | Not mentioned | RT-qPCR Digital drop PCR | 100% match to NPS positive samples (One sample was positive for saliva, but not for NPS.) |
| Azzi et al., 2020 | Saliva | ✓ | X | ✓ | ✓ |
| | PBS dilution | QIAmp Viral RNA mini kit | Not mentioned | RT-qPCR | 100% match to NPS |
| Byrne et al., 2020 | Saliva | ✓ | X | ✓ | ✓ |
| | | QIAamp Viral RNA Mini Kit | Not mentioned | RT-qPCR | 86% match to NPS |
| Chen et al., 2020 | Saliva | ✓ | X | ✓ | ✓ |
| | Mixed in VTM | Xpert Xpress SARS-CoV-2 assay | Not mentioned | RT-qPCR Xpert Xpress SARS-CoV-2 assay | 84% positive in both NPS and saliva, 10% positive in NPS only, and 5.2% positive in saliva only |
| Cheuk et al., 2020 | Saliva | ✓ | X | ✓ | ✓ |
| | | MagNA Pure LC 2.0 MagNA Pure 96 | Not mentioned | RT-qPCR | 85% match to NPS |
| Güçlü et al., 2020 | Saliva | ✓ | X | ✓ | ✓ |
| | | EZ1 Virus Kit Qiagen | Not mentioned | RT-qPCR | 85% match to NPS |
| Han et al., 2020 | Saliva | ✓ | ✓ | ✓ | ✓ |
| | | Seegene platform | Positivity decreased to 33% and 11%, at week 2 and 3, respectively. | RT-qPCR | 80% match to NPS |
| Hanson et al., 2020 | Saliva | ✓ | ✓ | ✓ | ✓ |
| | Diluted in ARUP laboratories transport medium | Hologic Aptima Panther platform | 5 days at 4° C. & Room Temperature | RT-qPCR | 94% match to NPS |
| Iwasaki et al., 2020 | Saliva | ✓ | X | ✓ | ✓ |
| | Mixed with PBS | QIAamp Viral RNA Mini Kit | Not mentioned | RT-qPCR | 100% match to NPS |
| Kojima et al., 2020 | Saliva | ✓ | X | ✓ | ✓ |
| | DNA/RNA Shield™ solution | RNA purification kit, Norgen Biotek Corp | Not mentioned | RT-qPCR | Physician supervised: 90% match to NPS Self collected: 66% match to NPS |
| Leung et al., 2020 | Saliva | ✓ | X | ✓ | ✓ |
| | Mixed in VTM | MagMAX viral RNA isolation kit | Not mentioned | RT-qPCR | 79% match to NPS |
| Mao et al., 2020 | Saliva | X | X | ✓ | ✓ |
| | | Not mentioned | Not mentioned | RT-qPCR | Saliva alone had a 74% match to NPS, but if there was sputum, then match increased to 93% |
| McCormick-Baw et al., 2020 | Saliva | ✓ | X | ✓ | ✓ |
| | | Cepheid Xpert Xpress SARS-CoV-2 assay | Not mentioned | RT-qPCR | 96% match to NPS |
| Migueres et al., 2020 | Saliva | ✓ | X | ✓ | ✓ |
| | | Hologic Aptima Panther platform | Not mentioned | RT-qPCR | 88% and 95% match for asymptomatic and symptomatic |
| | | | | | patients, respectively |
| Pasomsub et al., 2020 | Saliva | ✓ | X | ✓ | ✓ |
| | UTM added | bioMerieux lysis buffer MagDEA® Dx platform | Not mentioned | RT-qPCR | 98% match to NPS |
| Rao et al., 2020 | Saliva | ✓ | X | ✓ | ✓ |
| | | MagNA Pure 96 DNA and Viral NA Small Volume extraction kit | Not mentioned | RT-qPCR | SARS-CoV-2 was detected more frequently using saliva (93%) than NPS (52%) |
| Senok et al., 2020 | Saliva | ✓ | X | ✓ | ✓ |
| | | Chemagic™ 360 Nucleic Acid Extractor | Not mentioned | Rt-qPCR | 73% match to NPS |
| To et al., 2020 | Saliva | ✓ | X | ✓ | ✓ |
| | VTM added | NucliSENS easyMAG | Not mentioned | RT-qPCR | 92% match to NPS |
| Uwamino et al., 2020 | Saliva | X | ✓ | ✓ | ✓ |
| | | Not mentioned | Room temperature for 7 days | RT-qPCR | 32 positive by both NPS and saliva, 15 by NPS only, 11 by saliva only |
| Wong et al., 2020 | Saliva | ✓ | X | ✓ | ✓ |
| | | MagNA Pure LC 2.0 MagNA Pure 96 | Not mentioned | RT-qPCR | 85% match to NPS |
| Wyllie et al., 2020 | Saliva | ✓ | ✓ | ✓ | ✓ |
| | | MagMAX Viral/Pathogen Nucleic Acid Isolation kit | Mentioned stable for 25 days at Room Temperature, but not tested | RT-qPCR | 81% were positive by saliva and 71% by NPS |
| Vaz et al., 2020 | Saliva | ✓ | X | ✓ | ✓ |
| | Diluted in PBS | QIAGEN QIAamp® RNA Mini Kit | Not mentioned | RT-qPCR | 96% match to NPS |
| Yokota et al., 2021 | Saliva | ✓ | X | ✓ | ✓ |
| | Diluted in PBS | QIAsymphony DSP Virus/Pathogen kit and QIAamp Viral RNA Mini Kit | Not mentioned | RT-qPCR LAMP | 86% sensitivity for NPS and 92% for saliva |

### References:

Altawalah, H.; Al Huraish, F.; Alkandari, W.A.; Ezzikouri, S. Saliva specimens for detection of severe acute respiratory syndrome coronavirus 2 in Kuwait: a cross-sectional study. J. Clin. Virol. 2020, 132, 104652. DOI: HYPERLINK “https://d.docs.live.net/26efea6a3c49de6b/Documents/Saliva SARS-COV-2/Manuscript/10.1016/j.jcv.2020.104652″10.1016/j. jcv.2020.104652
Aita, A.; Basso, D.; Cattelan, A.M.; Fioretto, P.; Navaglia, F.; Barbaro, F.; Stoppa, A.; Coccorullo, E.; Farella, A.; Socal, A.; Vettor, R.; Plebani, M. SARS-CoV-2 identification and IgA antibodies in saliva: one sample two tests approach for diagnosis. Clin. Chim. Acta. 2020, 510,717-722. DOI: HYPERLINK “https://d.docs.live.net/26efea6a3c49de6b/Documents/Saliva SARS-COV-2/Manuscript/h10.1016/j.cca.2020.09.018″h10.1016/j.cca.2020.09.018
Azzi, L.; Carcano, G.; Gianfagna, F.; Grossi, P.; Gasperina, D.D.; Genoni, A.; Fasano, M.; Sessa, F.; Tettamanti, L.; Maurino, V.; Carinci, F.; Rossi, A.; Tagliabue, A.; Baj, A. Saliva is a reliable tool to detect SARS-CoV-2. J. Infect. 2020, 81(1),e45-e50. DOI: HYPERLINK “https://d.docs.live.net/26efea6a3c49de6b/Documents/Saliva SARS-COV-2/Manuscript/10.1016/j. jinf.2020.04.005″10.1016/j. jinf.2020.04.005
Byrne, R.L.; Kay, G.A.; Kontogianni, K.; Aljayyoussi, G.; Brown, L.; Collins, A.M.; Cuevas, L.E.; Ferreira, D.M.; Fraser, A.J.; Garrod, G.; Hill, H.; Highes, G.L.; Menzies, S.; Mitsi, E.; Owen, S.I.; Patterson, E.I.; Williams, C.T.; Hyder-Wright, A.; Adams, E.R.; Cubas-Atienzar, A.l. Saliva alternative to upper respiratory swabs for SARS-CoV-2 diagnosis. Emerg. Infect. Dis. 2020, 26(11), 2769-2770. DOI: HYPERLINK “https://d.docs.live.net/26efea6a3c49de6b/Documents/Saliva SARS-COV-2/Manuscript/10.3201/eid2611.203283″1 0.3201/eid2611.203283
Chen, J.K.H.; Yip, C.C.Y.; Poon, R.W.S.; Chan, K.H.; Cheng, V.C.C.; Hung, I.F.N.; Chan, J.F.W.; Yeun, K.Y.; To, K.K.W. Evaluating the use of posterior oropharyngeal saliva in a point-of-care assay for the detection of SARS-CoV-2. Emerg. Microb. Infect. 2020, 9(1), 1356-1359. DOI: HYPERLINK “https://d.docs.live.net/26efea6a3c49de6b/Documents/Saliva SARS-COV-2/Manuscript/10.1080/22221751.2020.1775133″10.1080/22221751.2020.1775133
Güçlü, E.; Koroglu, M.; Yürümez, Y.; Toptan, H.; Kose, E.; Guneysu, F.; Karabay, O. Comparison of saliva and oro-nasopharyngeal swab sample in the molecular diagnosis of COVID-19. Rev. Assoc. Med. Bras. 2020, 66(8), 1116-1121. DOI: 10.1590/1806-9282.66.8.1116
Han, M.S.; Seong, M.W.; Kim, N.; Shin, S.; Cho, S.I.; Park, H.; Kim, T.S.; Park, S.S.; Choi, E.H. Viral RNA load in mildly symptomatic and asymptomatic children with COVID-19, Seoul, South Korea. Emerg. Infect. Dis. 2020, 26(10),2497-2499. DOI: HYPERLINK “https://d.docs.live.net/26efea6a3c49de6b/Documents/Saliva SARS-COV-2/Manuscript/10.3201/eid2610.202449″10.3201/eid2610.202449
Hanson, K.E.; Barker, A.P.; Hillyard, D.R.; Gilmore, N.; Barrett, J.W.; Orlandi, R.R.; Shakir, S.M. Self-collected anterior nasal and saliva specimens versus healthcare worker-collected nasopharyngeal swabs for the molecular detection of SARS-CoV-2. J. Clin. Microbiol. 2020, 58(11), e01824-20. DOI: HYPERLINK “https://d.docs.live.net/26efea6a3c49de6b/Documents/Saliva SARS-COV-2/Manuscript/10.1128/JCM.01824-20″10.1128/JCM.01824-20
Iwasaki, S.; Fujisawa, S.; Nakakubo, S.; Kamada, K.; Yamashita, Y.; Fukumoto, T.; Sato, K.; Oguri, S.; Taki, K.; Senjo, H.; Sugita, J.; Hayasaka, K.; Konno, S.; Nishida, M.; Teshima, T. Comparison of SARS-CoV-2 detection in nasopharyngeal swab and saliva. J. Infect. 2020, 81(2), e145-e147. DOI: HYPERLINK “https://d.docs.live.net/26efea6a3c49de6b/Documents/Saliva SARS-COV-2/Manuscript/10.1016/j jinf.2020.05.071″10.1016/j.jinf.2020.05.071
Kojima, N.; Turner, F.; Slepnev, V.; Bacelar, A.; Deming, L.; Kodeboyina, S.; Klausner, J. D. Self-collected oral fluid and nasal swabs demonstrate comparable sensitivity to clinician collected nasopharyngeal swabs for coronavirus disease 2019 detection. Clin. Infect. Dis. 2021, 73(9), e3106-e3109. DOI: 10.1093/cid/ciaa1589
Leung, E.C.; Chow, V.C.; Lee, M.K.; Lai, R.W. Deep throat saliva as an alternative diagnostic specimen type for the detection of SARS-CoV-2. J. Med. Virol. 2021, 93(1), 533-536. DOI: HYPERLINK “https://d.docs.live.net/26efea6a3c49de6b/Documents/Saliva SARS-COV-2/Manuscript/10.1002/jmv.26258″10.1002/jmv.26258
Mao, M.H.; Guo, J.J.; Qin, L.Z.; Han, Z.X.; Wang, Y.J.; Yang, D. Serial semiquantitative detection of SARS-CoV-2 in saliva samples. J. Infect. 2021, 82(3), 414-451. DOI: HYPERLINK “https://d.docs.live.net/26efea6a3c49de6b/Documents/Saliva SARS-COV-2/Manuscript/10.1016/j.jinf.2020.10.002″10.1016/j.jinf.2020.10.002
Mccormick-Baw, C.; Morgan, K.; Gaffney, D.; Cazares, Y.; Jaworski, K.; Byrd, A.; Molberg, K.; Cavuoti, D. Saliva as an alternate specimen source for detection of SARS-CoV-2 in symptomatic patients using cepheid xpert xpress SARS-CoV-2. J. Clin. Microbiol. 2020, 58(8), e01109-20. DOI: 10.1128/JCM.01109-20
Migueres, M.; Mengelle, C.; Dimeglio, C.; Didier, A.; Alvarez, M.; Delobel, P.; Mansuy, J.M.; Izopet, J. Saliva sampling for diagnosing SARS-CoV-2 infections in symptomatic patients and asymptomatic carriers. J. Clin. Virol. 2020, 130, 104580. DOI: HYPERLINK “https://d.docs.live.net/26efea6a3c49de6b/Documents/Saliva SARS-COV-2/Manuscript/10.1016/j.jcv.2020.104580″10.1016/j.jcv.2020.104580
Pasomsub, E.; Watcharananan, S.P.; Boonyawat, K.; Janchompoo, P.; Wongtabtim, G.; Suksuwan, W.; Sungkanuparph, S.; Phuphuakrat, A. Saliva sample as a non-invasive specimen for the diagnosis of coronavirus disease 2019 (COVID-19): a cross-sectional study. Clin. Microbiol. Infect. 2021, 27(2), 285.e1-285.e4. DOI: 10.1016/ j.cmi.2020.05.001
Rao, M.; Rashid, F.A.; Sabri, F.S.A.H.; Jamil, N.N.; Zain, R.; Hasim, R.; Amran, F.; Kok, H.T.; Samad, M.A.; Ahmad, N. Comparing nasopharyngeal swab and early morning saliva for the identification of SARS-CoV-2. Clin. Infect. Dis. 2021, 72(9), e352-e356. DOI: 10.1093/cid/ciaa1156
Senok, A.; Alsuwaidi, H.; Atrah, Y.; Ayedi, O.A.; Zahid, J.A.; Han, A.; Al Marzooqi, A.; Heialy, S.A.; Altrabulsi, B.; AbdelWareth, L.; Idaghdour, Y.; Ali, R.; Loney, T.; Alsheikh-Ali, A. Saliva as an alternative specimen for molecular COVID-19 testing in community settings and population-based screening. Infect. Drug. Resist. 2020, 13, 3393-3399. DOI: HYPERLINK “https://d.docs.live.net/26efea6a3c49de6b/Documents/Saliva SARS-COV-2/Manuscript/10.2147/IDR.S275152″10.2147/IDR.S275152
To, K.K.W.; Tsang, O.T.Y.; Yip, C.C.Y.; Chan, K.H.C.; Wu, T.C.; Chan, J.M.C.; Leung, W.S.; Chik, T.S.H.; Choi, C.Y.C.; Kandamby, D.H.; Lung, D.C.; Tam, A.R.; Poon, R.W.S.; Fung, A.Y.F.; Hung, I.F.N.; Cheng, V.C.C.; Chan, J.F.W.; Yuen, K.Y. Consistent Detection of 2019 Novel Coronavirus in Saliva. Clin. Infect. Dis. 2020, 71(15), 841-843. DOI: HYPERLINK “https://d.docs.live.net/26efea6a3c49de6b/Documents/Saliva SARS-COV-2/Manuscript/10.1093/cid/ciaa149″10.1093/cid/ciaa149
Uwamino, Y.; Nagata, M.; Aoki, W.; Fujimori, Y.; Nakagawa, T.; Yokota, H.; Sakai-Tagawa, Y.; Iwatsuki-Horimoto, K.; Shiraki, T.; Uchida, S.; Uno, S.; Kabata, H.; Ikemura, S.; Kamata, H.; Ishii, M.; Fukunaga, K.; Kawaoka, Y.; Hasegawa, N.’ Mitsuru, M. Accuracy and stability of saliva as a sample for reverse transcription PCR detection of SARS-CoV-2. J. Clin. Pathol. 2020, 74(1), 67-68. DOI: HYPERLINK “https://d.docs.live.net/26efea6a3c49de6b/Documents/Saliva SARS-COV-2/Manuscript/10.1136/jclinpath-2020-206972″10.1136/jclinpath-2020-206972
Wong, S.C.; Tse, H.; Siu, H.K.; Kwong, T.S.; Chu, M.Y.; Yau, F.Y.S.; Cheung, I.Y.Y.;
Tse, C.W.Z.; Poon, K.C.; Cheung, K.C.; Wu, T.C.; Chan, J.W.M.; Cheuk, W.; Lung,
D.C. Posterior oropharyngeal saliva for the detection of SARS-CoV-2. Clin. Infect.
Dis. 2020, 71(11), 2939-2946. DOI: 10.1093/cid/ci aa797
Wyllie, A.L.; Fournier, J.; Casanovas-Massana, A. Saliva is more sensitive for SARS-CoV-2 detection in COVID-19 patients than nasopharyngeal swabs. N. Engl. J. Med. 2020, 383,1283-1286. DOI: 10.1056/NEJMc2016359
Vaz, S.N.; Santana, D.S.; Netto, E.M.; Pedroso, C.; Wang, W.K.; Santos, F.D.A.; Brites, C. Saliva is a reliable, non-invasive specimen for SARS-CoV-2 detection. Braz. J. Infect. Dis. 2020, 24(5), 422-427. DOI: HYPERLINK “https://d.docs.live.net/26efea6a3c49de6b/Documents/Saliva SARS-COV-2/Manuscript/10.1016/j.bjid.2020.08.001″10.1016/j.bjid.2020.08.001
Yokota, I.; Shane, P.Y.; Okada, K.; Unoki, Y.; Yang, Y.; Inao, T.; Sakamaki, K.; Iwasaki, S.; Hayasaka, K.; Sugjita, J.; Nishida, M.; Fujisawa, S.; Teshima, T. Mass screening of asymptomatic persons for SARS-CoV-2 using saliva. Clin. Infect. Dis. 2021, 73(3), e559-e565. DOI: HYPERLINK “https://d.docs.live.net/26efea6a3c49de6b/Documents/Saliva SARS-COV-2/Manuscript/10.1093/cid/ciaa1388″10.1093/cid/ciaa1388

Table 3. A summary of studies on the detection of SARS-CoV-2 in gargle. Inactivation/extraction method, RNA stability, detection method, and sensitivity comparison to nasopharyngeal swabs (NPS) analysis are summarized. The checkmarks indicate that the study addressed certain aspects.

TABLE 3

| | | | | | |
|---|---|---|---|---|---|
| Study | Sample | Inactivation/Extraction | RNA Stability | Detection Method | Sensitivity/Comparison to NPS positive samples |
| Goldfarb et al., 2021 | Gargle | ✓ | ✓ | ✓ | ✓ |
| | Saline solution | QiaSymphony automated extractor using the DSP virus/pathogen minikit Cepheid Xpert Xpress SARS-CoV-2 assay | 2 days at Room Temperature | RT-qPCR Cepheid Xpert Xpress SARS-CoV-2 assay | 98% sensitivity, 39/40 NPS confirmed patients tested positive using gargle |
| Kandel et al., 2021 | Gargle | ✓ | X | ✓ | ✓ |
| | Saline solution | Heated at 56° C. for 30 mins in a dry bath filled with thermal beads and vortexed for 30 secs. TNA lysis buffer (plus carrier RNA, and MS2 phage internal control) MagBind Viral RNA Xpress kit | Not mentioned | RT-qPCR | 90% match to NPS |
| Lopez-Lopes et al., 2020 | Gargle | ✓ | X | | ✓ |
| | Saline solution | Automated extraction (Bio Gene, Quibasa or Abbott M2000) | Not mentioned | RT-qPCR | Not all samples had a paired NPS, but study generally found that Ct values of throat washes were comparable to NPS but higher |
| Malecki et al., 2021 | Gargle | ✓ | X | ✓ | ✓ |
| | Saline solution | Not mentioned | Not mentioned | RT-qPCR | Screened 924 healthcare workers, 26 were positive |
| Paré et al., 2021 | Gargle | ✓ | X | ✓ | ✓ |
| | Natural spring water | Proteinase K (PK) & heated at 56° C. for10 min Thermal lysis: 90° C. for 2 min | Not mentioned | In house laboratory developed (LD) NAAT | 1297 adult samples processed. Overall sensitivity was 98% for NPS and 90% for gargles. |
| Poukka et al., 2021 | Both | ✓ | X | ✓ | ✓ |
| | Saliva & Gargle | Viscous samples were diluted with PBS and vortexed Chemagic Viral300 DNA/RNA kit H96 | Not mentioned | One step RT-qPCR | Saliva had 100% sensitivity Gargle had 97% sensitivity |

References:
Goldfarb, D.M.; Tilley, P.; Al-Rawahi, G.N.; Srigley, J.A.; Ford, G.; Pedersen, H.; Pabbi, A.; Hannam-Clark, S.; Charles, M.; Dittrick, M.; Gadkar, V.J.; Pernica, J.M.; Hoang, L.M.N. Self-Collected Saline Gargle Samples as an Alternative to Health Care Worker-Collected Nasopharyngeal Swabs for COVID-19 Diagnosis in Outpatients. J. Clin. Microbiol. 2021, 59(4), e02427-20. DOI: 10.1128/JCM.02427-20
Kandel, C.E.; Young, M.; Serbanescu, M.A.; Powis, J.E.; Bulir, D.; Callahan, J.; Katz, K.; McCready, J.; Racher, H.; Sheldrake, E.; Quon, D.; Vojdani, O.K.; McGeer, A.; Goneau, L.W.; Vermeiren, C. Detection of severe acute respiratory coronavirus virus 2 (SARS-CoV-2) in outpatients: A multicenter comparison of self-collected saline gargle, oral swab, and combined oral-anterior nasal swab to a provider collected nasopharyngeal swab. Infect. Control Hosp. Epidemio. 2021, 42 (11), 1340-1344. DOI: 10.1017/ice.2021.2
Lopez-Lopes, G.I.; Ahagon, C.; Benega, M.A.; da Silva, D.B.B.; Silva, V.O.; Santos, K.C.D.O.; do Prado, K.S.D.P.; dos Santos, F.P.; Cilli, A.; Saraceni, C.; da Cruz, N.B.; Afonso, A.M.S.; Timenetsky, M.D.C.; de Macedo Brigido, L. Throat wash as a source of SARS-CoV-2 RNA to monitor community spread of COVID-19. medRxiv. 2020. DOI: HYPERLINK “https://d.docs.live.net/26efea6a3c49de6b/Documents/Saliva SARS-COV-2/Manuscript/10.1101/2020.07.29.20163998″10.1101/2020.07.29.20163998 (accessed 2021 Dec 10).
Malecki, M.; Lüsebrink, J.; Teves, S.; Wendel, A.F. Pharynx gargle samples are suitable for SARS-CoV-2 diagnostic use and save personal protective equipment and swabs. Infect. Control Hosp. Epidemiol. 2021, 42(2), 248-249. DOI: 10.1017/ice.2020.229
Paré, S.G.; Bestman-Smith, J.; Fafard, J.; Doualla-Bell, F.; Jacob-Wagner, M.; Lavallée, C.; Charest, H.; Beauchemin, S.; Coutlée, F.; Dumaresq, J.; Busque, L.; St-Hilaire, M.; Lepine, G.; Boucher, V.; Desforges, M.; Goupil-Sormany, I.; Labbe, A.C. Natural spring water gargle samples as an alternative to nasopharyngeal swabs for SARS-CoV-2 detection using a laboratory-developed test. J. Med. Virol. 2021. DOI: 10.1002/jmv.27407
Poukka, E.; Mäkelä, H.; Hagberg, L.; Vo, T.; Nohynek, H.; Ikonen, N.; Liitsola, K.; Helve, O.; Savolainen-Kopra, C.; Dub, T. Detection of SARS-CoV-2 Infection in Gargle, Spit, and Sputum Specimens. Microbiol. Spectr. 2021, 9(1), e00035-21. DOI: 10.1128/Spectrum.00035-21

Table 4. A summary of reported methods that were used to detect SARS-CoV-2 in pooled samples. The checkmarks indicate that the study addressed certain aspects.

TABLE 4

| | | | | | |
|---|---|---|---|---|---|
| Study | Sample | Inactivation/Extrac tion | RNA Stability | Detection Method | Results |
| VIP-Mag Method | Both | ✓ | X | ✓ | ✓ |
| | Saliva | Single Tube Method (sample collection, inactivation, lysis, & maintains RNA stability) Heat at 55° C., 10 min | 1 week at 4° C. & Room Temperature (RNA) Directly inputted into RT-qPCR without the need for elution/purificati on | RT-qPCR | Positive detectable even after diluting by 32 times |
| Barat et al., 2021 | Saliva | ✓ | X | ✓ | ✓ |
| | | Proteinase K, vortexed and heated for 5 min at 95° C. | Not mentioned | RT-qPCR | 90-94% sensitivity in 5 pooled samples |
| | | NucliSENS easyMAG Panther Fusion Cobas 6800 | | | |
| Bokelmann et al., 2021 | Gargle | ✓ | X | ✓ | ✓ |
| | Sterile water | Lysis/binding buffer (Tris-HCl, LiCl, LiDS, EDTA, DTT) Quick extract (Lucigen) | Not mentioned | RT-qPCR capture and improved loop-mediated isothermal amplification (CAP-LAMP) | 1 positive in 25 pooled samples can be detected |
| Kellner et al., 2021 | Gargle | ✓ | X | ✓ | ✓ |
| | Saline solution Hank’s balanced salt solution | Guanidine thiocyanate KingFisher magnetic particle processor DNasel for 15 mins at 37° C. QuickExtract DNA extraction solution (Lucigen) | Not mentioned | RT-qPCR LAMP | 1 positive in 100 pooled samples can be detected |
| Willeit et al., 2021 | Gargle | ✓ | X | ✓ | ✓ |
| | Saline solution or a modified Hank’s balanced salt solution 2 M 1,4-dithiothrei tol added to reduce viscosity | 10 pooled samples mixed using KingFisher Flex mixer Lysis buffer (Tris, GITC, EDTA, 2% Triton X-100, DTT) added and incubated for 10 min at room temperature KingFisher Flex Magnetic Particle Processor System | Not mentioned | RT-qPCR | This method was used to screen 10 734 participants from 245 schools in Austria. |
| | | Carboxylated magnetic bead CyBio Felix System | | | |

References:
Barat, B.; Das, S.; De Giorgi, V.; Henderson, D.K.; Kopka, S.; Lau, A.F.; Miller, T.; Moriarty, T.; Palmore, T.N.; Sawney, S.; Spalding, C.; Tanjutco, P.; Wortmann, G.; Zelazny, A.M.; Frank, K.M. Pooled saliva specimens for SARS-CoV-2 testing. J. Clin. Microbiol. 2021, 59(3), e02486-20. DOI: 10.1128/JCM.02486-20
Bokelmann, L.; Nickel, O.; Maricic, T.; Pääbo, S.; Meyer, M.; Borte, S.; Riesenberg, S. Point-of-care bulk testing for SARS-CoV-2 by combining hybridization capture with improved colorimetric LAMP. Nat. Commun. 2021, 12(1), 1-8. DOI: 10.1038/s41467-021-21627-0
Kellner, M.J.; Ross, J.J.; Schnabl, J.; Dekens, M.P.; Heinen, R.; Grishkovskaya, I.; Bauer, B.; Stadlmann, J.; Menéndez-Arias, L.; Fritsche-Polanz, R.; Traugott, M.; Seitz, T.; Zoufaly, A.; Fodinger, M.; Wenisch, C.; Zuber, J.; Pauli, A.; Brennecke, J. A rapid, highly sensitive and open-access SARS-CoV-2 detection assay for laboratory and home testing. bioRxiv. 2020. DOI: HYPERLINK “https://d.docs.live.net/26efea6a3c49de6b/Documents/Saliva SARS-COV-2/Manuscript/10.1101/2020.06.23.166397″10.1101/2020.06.23.166397 (accessed 2021 Dec 10).
Willeit, P.; Krause, R.; Lamprecht, B.; Berghold, A.; Hanson, B.; Stelzl, E.; Stoiber, H.; Zuber, J.; Heinen, R.; Köhler, A.; Bernhard D.; Borena, W.; Doppler, C.; von Laer, D.; Schmidt, H.; Proll, J.; Steinmetz, I.; Wagner, M. Prevalence of RT-qPCR-detected SARS-CoV-2 infection at schools: First results from the Austrian School-SARS-CoV-2 prospective cohort study. The Lancet Regional Health-Europe. 2021, 5, 100086. DOI: 10.1016/j.lanepe.2021.100086

### Comparison of Tap Water and Saline Used for Collecting Gargle Samples

To explore whether tap water is suitable for collecting a gargle sample, we compared the stability of SARS-CoV-2 viral RNA in tap water gargle and saline gargle samples. We obtained pooled tap water gargle and pooled saline gargle from SARS-CoV-2 negative volunteers. We added 65, 390, or 3900 copies of viral RNA to each type of the pooled gargle samples. We analyzed these samples after they were stored at room temperature for 2 h. The results show that the tap water gargles containing 65 or 390 copies of the viral RNA required higher threshold cycles (Ct) to achieve detection than those for their saline gargle counterparts (FIG. 9A). We also tested three saline gargle and three tap water gargle samples collected from a SARS-CoV-2 positive patient (FIG. 9B). The Ct values are consistently higher for the tap water gargle samples than for the saline gargles samples. These results indicate lower concentrations of the viral RNA in tap water gargle than in saline gargle samples, probably because of more degradation of the viral RNA in tap water. Saline was used for the subsequent collection of all the gargle samples in this study.

### Additional Information on Enhancing the Recovery of Low Amounts of RNA.

We compared the use of a commercially available RNA carrier and glycogen for enhancing the recovery of low amounts of RNA from gargle samples. We added 17, 34, or 68 ng/ µL of glycogen or 1.7 ng/µL of Carrier RNA (1 µg per sample, recommended by MagMAX viral RNA isolation kit) in VIP buffer. We mixed 600 µL of VIP buffer with 200 µL of gargle samples containing 390 or 3900 copies of viral RNA. As shown in FIG. 10, the addition of glycogen reduced the threshold cycles (Ct) needed for the detection of 390 copies of viral RNA, suggesting a better recovery of the viral RNA for detection. The effect of “Carrier RNA” and glycogen on the higher concentration (3900 copies) of viral RNA is minimum. To achieve efficient recovery of minute amounts of viral RNA from the samples, we added glycogen into VIP buffer to a final concentration of 17 ng/µL.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of detecting SARS-CoV-2, or variants thereof, in a sample, comprising:
i) combining a sample with a buffer to obtain a mixture, the buffer comprising: a chaotropic agent, a reducing agent, a surfactant, and a serine protease,
ii)- heating said mixture to obtain a heated mixture;
iii)- subjecting the heated mixture to a centrifugation step to obtain a supernatant comprising RNA;
iiii) combining the supernatant with a magnetic bead suspension, such that at least a portion of the RNA within the supernatant binds to the magnetic beads, the magnetic bead suspension obtained by suspension of magnetic beads in a bead-binding buffer to obtain RNA-bound magnetic beads,
(v) washing said RNA-bound magnetic beads to obtained washed beads; and
(vi) conducting a reverse-transcription polymerase chain reaction (RT-PCR) assay on the washed beads using primers to amplify SARS-CoV-2 or variants thereof.

2. The method of claim 1, wherein said chaotropic agent is guanidinium isothiocyanate, said reducing agent is mercaptoethanol, said surfactant is Triton X-100, and/or said serine protease is proteinase K.

3. The method of claim 1, wherein (i) said buffer comprises about 6 M guanidinium isothiocyanate, about 3% 2-mercaptoethanol, about 2.5% Triton X-100, and about 170 ng/µL proteinase K.

4. The method of claim 1, wherein (i) said buffer further comprises about 17 ng/µL glycogen.

5. The method of claim 1, wherein said bead-binding buffer comprises about 20 mM Tris-HCl pH 8.0, about 2 M NaCl, about 36% PEG 8000, and about 2 mM EDTA.

6. The method of claim 1, wherein (ii) heating said mixture comprises said mixture at 55° C. for about 10 minutes.

7. The method of claim 1, wherein (iii) said centrifugation step comprises subjecting the heated mixture to a centrifugation of 13000xg for 5 minutes.

8. The method of claim 1, in (iiii) further comprising washing said RNA-bound magnetic beads, comprising:
adding ethanol to said RNA-bound magnetic beads mixing for about 10 seconds at room temperature,
collecting said RNA-bound magnetic beads by centrifugation at 13000xg for about 2 minutes to obtain collected RNA-bound magnetic beads,
washing said collected RNA-bound magnetic beads with 75% ethanol followed by air drying for 5 minutes to obtain dried RNA-bound magnetic beads, and
resuspending said dried RNA-bound magnetic beads RNase-free water containing 200 ng/µLof Proteinase K inhibitor.

9. The method of claim 1, wherein the sample is saliva.

10. The method of claim 1, wherein the sample is a gargle solution using water or saline.

11. The method of claim 1, wherein the sample is from a human.

12. An RNA Preservation buffer, comprising:
a chaotropic agent, a reducing agent, a surfactant, and a serine protease.

13. The RNA Preservation buffer of claim 12, wherein said chaotropic agent is guanidinium isothiocyanate, said reducing agent is mercaptoethanol, said surfactant is Triton X-100, and/or said serine protease is proteinase K.

14. The RNA Preservation buffer of claim 12, wherein said buffer comprises 6 M guanidinium isothiocyanate, 3% 2-mercaptoethanol, 2.5% Triton X-100, 170 ng/µL proteinase K.

15. The RNA Preservation buffer of claim 12, wherein said buffer further comprises about 17 ng/µL glycogen.

16. A Kit for detecting SARS-CoV-2, or variants thereof, in a sample, comprising:
a) 6 M guanidinium isothiocyanate, 3% 2-mercaptoethanol, 2.5% Triton X-100, 170 ng/µLof proteinase K, and 17 ng/µLof glycogen, and
b) a container.

17. The kit of claim 16 further comprising a bead-binding buffer comprising 20 mM Tris-HCl pH 8.0, 2 M NaCl, 36 % PEG-8000, and 2 mM EDTA.
